(19) United States
(12) Patent Application Publication
(10) Pub. No.: US 2007/0254952 A1
(43) Pub. Date: Nov. 1, 2007

(54) CANNABINOID RECEPTOR MODULATORS

(76) Inventors: Yuguang Wang, Monroe, NJ (US); Samuel Chackalamannil, Califon, NJ (US); Yuriko Y. Root, Old Bridge, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/736,722

(22) Filed: Apr. 18, 2007

Related U.S. Application Data
(60) Provisional application No. 60/794,167, filed on Apr. 21, 2006.

(51) Int. Cl. A61K 31/277; A61K 31/138; A61K 31/075

(52) U.S. Cl. 514/520; 514/719; 514/649; 558/423; 564/338; 568/649

(57) ABSTRACT

Disclosed are compounds of the formula: 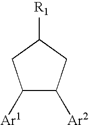
or the pharmaceutically acceptable salts or solvates thereof, wherein: Ar¹ is a chlorophenyl group, Ar² is a dichlorophenyl group, R¹ is a —(CH₂)ₘ—X—(CH₂)ₙ—R² group, X is a —NH—, —O—, —C(O)— or —S(O)₂— group, and R² is a substituted phenyl group. These compounds are CB1 receptor modulators. Also disclosed are methods of treating CB1 modulated diseases or conditions such as the metabolic syndrome.

### REFERENCE TO RELATED APPLICATION

This application claims the benefit of U.S. Provisional Application Ser. No. 60/794167 filed Apr. 21, 2006.

### FIELD OF THE INVENTION

The present invention relates to cannabinoid receptor modulators, particularly, antagonists or inverse agonists of the CB₁ receptor, useful for the treatment of obesity, metabolic disorders, addiction, diseases of the central nervous system, cardiovascular disorders, respiratory disorders, and gastrointestinal disorders, pharmaceutical compositions comprising such compounds, and methods of treatment using the compounds and compositions to treat conditions such as obesity, metabolic disorders, addiction, diseases of the central nervous system, cardiovascular disorders, respiratory disorders, and gastrointestinal disorders.

### BACKGROUND OF THE INVENTION

The CB₁ receptor is one of the most abundant neuromodulatory receptors in the brain, and is expressed at high levels in the hippocampus, cortex, cerebellum, and basal ganglia (e.g., Wilson et al., Science, 2002, vol. 296, 678-682). Selective CB₁ receptor antagonists, for example pyrazole derivatives such as rimonabant (e.g., U.S. Pat. No. 6,432,984), can be used to treat various conditions, such as obesity and metabolic syndrome (e.g., Bensaid et al., Molecular Pharmacology, 2003 vol. 63, no. 4, pp. 908-914; Trillou et al., Am. J. Physiol. Regul. Integr. Comp. Physiol. 2002 vol. 284, R345-R353; Kirkham, Am. J. Physiol. Regut. Integr. Comp. Physiol. 2002 vol. 284, R343-R344), neuroinflammatory disorders (e.g., Adam, et al., Expert Opin. Ther. Patents, 2002, vol. 12, no. 10, 1475-1489; U.S. Pat. No. 6,642,258), cognitive disorders and psychosis (e.g., Adam et al., Expert Opin. Ther. Pat., 2002, vol. 12, pp. 1475-1489), addiction (e.g., smoking cessation; U.S. Patent Publ. 2003/0087933), gastrointestinal disorders (e.g., Lange et al., J. Med. Chem. 2004, vol. 47, 627-643) and cardiovascular conditions (e.g., Porter et al., Pharmacology and Therapeutics, 2001 vol. 90, 45-60; Sanofi-Aventis Publication, Bear Stearns Conference, New York, Sep. 14, 2004, pages 19-24).

However, there is still a need for improved cannabinoid agents, particularly cannabinoid receptor modulators (e.g., antagonists or inverse agonists of the CB₁ receptor) with fewer side-effects and improved efficacy. It is therefore an object of the present invention to provide substituted cyclopentane rings that are cannabinoid receptor modulators useful in the treatment of diseases or conditions mediated by cannabinoid receptors.

### SUMMARY OF THE INVENTION

This invention provides compounds of formula 1 that are cannabinoid receptor modulators. The compounds of this invention are CB1 receptor modulators. Thus, this invention provides compounds of formula 1: 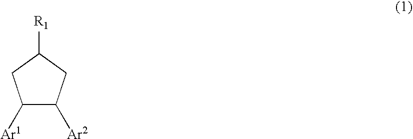
or the pharmaceutically acceptable salts thereof wherein: Ar¹ is a halo substituted linking group (e.g., —NH—, —O—, —C(O)— and —S(O)₂—), R² is substituted phenyl, m is 0 to 4, and n is 0 or 1.

This invention also provides compounds of formula 1.

This invention also provides pharmaceutically acceptable salts of the compounds of formula 1.

This invention also provides solvates of the compounds of formula 1.

This invention also provides the compounds 1A, 1B, 1C, 1D, and 1E.

This invention also provides the compounds 1A, 1B, 1C, and 1E.

This invention also provides the compound 1B.

This invention also provides compounds of formula 1 (e.g., any of the compounds described above) in pure form.

This invention also provides compounds of formula 1 (e.g., any of the compounds described above) in isolated form.

This invention also provides compounds of formula 1 (e.g., any of the compounds described above) in pure and isolated form.

This invention also provides a pharmaceutical composition comprising a therapeutically effective amount of at least one (e.g., 1, 2 or 3, or 1 or 2, or 1, and usually 1) compound of formula 1, or a pharmaceutically acceptable salt thereof, and a pharmaceutically acceptable carrier.

This invention also provides a pharmaceutical composition comprising a therapeutically effective amount of at least one (e.g., 1, 2 or 3, or 1 or 2, or 1, and usually 1) compound of formula 1, or a pharmaceutically acceptable salt thereof, at least one (e.g., 1, 2 or 3, or 1 or 2, or 1) other pharmaceutically active ingredient (such as the other pharmaceutically active ingredients (e.g., agents or drugs) described herein), and a pharmaceutically acceptable carrier.

This invention also provides a pharmaceutical composition comprising a therapeutically effective amount of at least one (e.g., 1, 2 or 3, or 1 or 2, or 1, and usually 1) compound of formula 1, or a pharmaceutically acceptable salt thereof at least one (e.g., 1, 2 or 3, or 1 or 2, or 1) cholesterol lowering agent (such as those cholesterol lowering agents described herein).

This invention also provides a method of treating a disease or a disorder (e.g., the metabolic syndrome) mediated by a cannabinoid receptor (e.g., CB1 receptor) in a patient in need of such treatment, said treatment comprising administering to said patient an effective amount of at least one (e.g., 1, 2 or 3, or 1 or 2, or 1) compound of formula 1.

This invention also provides a method of treating a disease or a disorder (e.g., the metabolic syndrome) mediated by a cannabinoid receptor (e.g., CB1 receptor) in a patient in need of such treatment, said treatment comprising administering to said patient an effective amount a compound of formula 1.

This invention also provides a method of treating a disease or disorder (e.g., the metabolic syndrome) mediated by a cannabinoid receptor (e.g., CB1 receptor) in a patient in need of such treatment, said treatment comprising administering to said patient an effective amount of at least one (e.g., 1, 2 or 3, or 1 or 2, or 1) compound of formula 1, and an effective amount of at least one (e.g., 1, 2 or 3, or 1 or 2, or 1) other pharmaceutically active ingredient (such as a pharmaceutically active ingredient previously used to treat said disease or disorder, such as the pharmaceutically active ingredients listed in the Physician's Desk Reference.

This invention also provides a method of treating a disease or disorder (e.g., the metabolic syndrome) mediated by a cannabinoid receptor (e.g., CB1 receptor) in a patient in need of such treatment, said treatment comprising administering to said patient an effective amount of a compound of formula 1, and an effective amount of another pharmaceutically active ingredient (such as a pharmaceutically active ingredient previously used to treat said disease or disorder, such as the pharmaceutically active ingredients listed in the Physician's Desk Reference.

This invention also provides a method of treating the metabolic syndrome in patient in need of such treatment, said treatment comprising administering to said patient at least one (e.g., 1, 2 or 3, or 1 or 2, or 1) compound of formula 1.

This invention also provides a method of treating the metabolic syndrome in patient in need of such treatment, said treatment comprising administering to said patient a compound of formula 1.

This invention also provides a method of treating the metabolic syndrome in a patient in need of such treatment, said treatment comprising administering to said patient an effective amount of at least one (e.g., 1, 2 or 3, or 1 or 2, or 1) compound of formula 1, and an effective amount of at least one (e.g., 1, 2 or 3, or 1 or 2, or 1) other pharmaceutically active ingredient (such as a pharmaceutically active ingredient previously used to treat said disease or disorder, such as the pharmaceutically active ingredients listed in the Physician's Desk Reference, such as a cholesterol lowering drug).

This invention also provides a method of treating the metabolic syndrome in a patient in need of such treatment, said treatment comprising administering to said patient an effective amount of a compound of formula 1, and an effective amount of at least one (e.g., 1, 2 or 3, or 1 or 2, or 1) other pharmaceutically active ingredient (such as a pharmaceutically active ingredient previously used to treat said disease or disorder, such as the pharmaceutically active ingredients listed in the Physician's Desk Reference, such as a cholesterol lowering drug).

This invention also provides a method of treating dyslipidemia (e.g., atherogenic dyslipidemia), in a patient in need of such treatment, said treatment comprising administering to said patient an effective amount of at least one (e.g., 1, 2 or 3, or 1 or 2, or 1) compound of formula 1. Thus, this invention provides a method treating high triglycerides, low HDL cholesterol and high LDL cholesterol, in a patient in need of such treatment, said treatment comprising administering to said patient an effective amount of at least one (e.g., 1, 2 or 3, or 1 or 2, or 1) compound of formula 1.

This invention also provides a method of treating dyslipidemia (e.g., atherogenic dyslipidemia), in a patient in need of such treatment, said treatment comprising administering to said patient an effective amount of a compound of formula 1. Thus, this invention provides a method treating high triglycerides, low HDL cholesterol and high LDL cholesterol, in a patient in need of such treatment, said treatment comprising administering to said patient an effective amount of a compound of formula 1.

This invention also provides a method of treating dyslipidemia (e.g., atherogenic dyslipidemia) in a patient in need of such treatment, said treatment comprising administering to said patient an effective amount of at least one (e.g., 1, 2 or 3, or 1 or 2, or 1) compound of formula 1, and an effective amount of at least one (e.g., 1, 2 or 3, or 1 or 2, or 1) other pharmaceutically active ingredient (such as a pharmaceutically active ingredient previously used to treat said disease or disorder, such as the pharmaceutically active ingredients listed in the Physician's Desk Reference, such as a cholesterol lowering drug). Thus, this invention also provides a a method of treating high triglycerides, low HDL cholesterol and high LDL cholesterol in a patient in need of such treatment, said treatment comprising administering to said patient an effective amount of at least one (e.g., 1, 2 or 3, or 1 or 2, or 1) compound of formula 1, and an effective amount of at least one (e.g., 1, 2 or 3, or 1 or 2, or 1) other pharmaceutically active ingredient (such as a pharmaceutically active ingredient previously used to treat said disease or disorder, such as the pharmaceutically active ingredients listed in the Physician's Desk Reference, such as a cholesterol lowering agent).

This invention also provides a method of treating dyslipidemia (e.g., atherogenic dyslipidemia) in a patient in need of such treatment, said treatment comprising administering to said patient an effective amount of a compound of formula 1, and an effective amount of at least one (e.g., 1, 2 or 3, or 1 or 2, or 1) other pharmaceutically active ingredient (such as a pharmaceutically active ingredient previously used to treat said disease or disorder, such as the pharmaceutically active ingredients listed in the Physician's Desk Reference, such as a cholesterol lowering drug). Thus, this invention also provides a a method of treating high triglycerides, low HDL cholesterol and high LDL cholesterol in a patient in need of such treatment, said treatment comprising administering to said patient an effective amount of a compound of formula 1, and an effective amount of at least one (e.g., 1, 2 or 3, or 1 or 2, or 1) other pharmaceutically active ingredient (such as a pharmaceutically active ingredient previously used to treat said disease or disorder, such as the pharmaceutically active ingredients listed in the Physician's Desk Reference, such as a cholesterol lowering agent).

This invention also provides a method of treating dyslipidemia (e.g., atherogenic dyslipidemia) in a patient in need of such treatment, said treatment comprising administering to said patient an effective amount of at least one (e.g., 1, 2 or 3, or 1 or 2, or 1) compound of formula 1, and an effective amount of at least one (e.g., 1, 2 or 3, or 1 or 2, or 1) cholesterol lowering agent.

Thus, this invention also provides a method of treating dyslipidemia (e.g., atherogenic dyslipidemia) in a patient in need of such treatment, said treatment comprising administering to said patient an effective amount of at least one (e.g., 1, 2 or 3, or 1 or 2, or 1) compound of formula 1, and an effective amount of at least one (e.g., 1, 2 or 3, or 1 or 2, or 1) cholesterol lowering agent selected from the group consisting of: ezetimibe (available as the Zetia® brand of ezetimibe), the combination of ezetimibe and simvastatin (available as the Vytorin® brand of ezetimibe/simvastatin), lovastatin (available as the Mevacor® brand of lovastatin), simvastatin (available as the Zocor® brand of simvastatin), pravastatin (available as the Pravachol® brand of pravastin), atorvastatin calcium (available as the Lipitor® brand of atorvastatin calcium), and rosuvastatin calcium (available as the Crestor® brand of rosuvastatin calcium). In one example of this method, the compound of formula 1 is administered with ezetimibe (available as the Zetia® brand of ezetimibe), and in another example of this method the compound of formula 1 is administered with ezetimibe/simvastatin (available as the Vytorin® brand of ezetimibe/simvastatin).

Thus, this invention also provides a method of treating high triglycerides, low HDL cholesterol and high LDL cholesterol in a patient in need of such treatment, said treatment comprising administering to said patient an effective amount of at least one (e.g., 1, 2 or 3, or 1 or 2, or 1) compound of formula 1, and an effective amount of at least one (e.g., 1, 2 or 3, or 1 or 2, or 1) cholesterol lowering agent selected from the group consisting of: ezetimibe (available as the Zetia® brand of ezetimibe), the combination of ezetimibe and simvastatin (available as the Vytorin® brand of ezetimibe/simvastatin), lovastatin (available as the Mevacor® brand of lovastatin), simvastatin (available as the Zocor® brand of simvastatin), pravastatin (available as the Pravachol® brand of pravastin), atorvastatin calcium (available as the Lipitor® brand of atorvastatin calcium), and rosuvastatin calcium (available as the Crestor® brand of rosuvastatin calcium). In one example of this method, the compound of formula 1 is administered with ezetimibe (available as the Zetia® brand of ezetimibe), and in another example of this method the compound of formula 1 is administered with the ezetimibe/simvastatin (available as the Vytorin® brand of ezetimibe/simvastatin).

This invention also provides a method of treating a disease or disorder mediated by a cannabinoid receptor (e.g., CB1 receptor) in a patient in need thereof, wherein said disease or disorder is selected from the group consisting of: neuroinflammatory disorders, cognitive disorders, psychosis, addictive behavior, gastrointestinal disorders, and cardiovascular conditions, said treatment comprising administering to said patient an effective amount of at least one (e.g., 1, 2 or 3, or 1 or 2, or 1, and usually 1) compound of formula 1.

This invention also provides a method of treating a disease or disorder mediated by a cannabinoid receptor (e.g., CB1 receptor) in a patient in need thereof, wherein said disease or disorder is selected from the group consisting of: neuroinflammatory disorders, cognitive disorders, psychosis, addictive behavior, gastrointestinal disorders, and cardiovascular conditions, said treatment comprising administering to said patient an effective amount of a compound of formula 1.

This invention also provides a method of treating a disease or disorder mediated by a cannabinoid receptor (e.g., CB1 receptor) in a patient in need thereof, wherein said disease or disorder is selected from the group consisting of: neuroinflammatory disorders, cognitive disorders, psychosis, addictive behavior, gastrointestinal disorders, and cardiovascular conditions, said treatment comprising administering to said patient an effective amount of at least one (e.g., 1, 2 or 3, or 1 or 2, or 1, and usually 1) compound of formula 1, in combination with at least one (e.g., 1, 2 or 3, or 1 or 2, or 1, or 1) other pharmaceutically active ingredient (such as a pharmaceutically active ingredient previously used to treat said disease or disorder, such as the pharmaceutically active ingredients listed in the Physician's Desk Reference for treating such diseases or disorders).

This invention also provides a method of treating a disease or disorder mediated by a cannabinoid receptor (e.g., CB1 receptor) in a patient in need thereof, wherein said disease or disorder is selected from the group consisting of: neuroinflammatory disorders, cognitive disorders, psychosis, addictive behavior, gastrointestinal disorders, and cardiovascular conditions, said treatment comprising administering to said patient an effective amount of a compound of formula 1, in combination with at least one (e.g., 1, 2 or 3, or 1 or 2, or 1, or 1) other pharmaceutically active ingredient (such as a pharmaceutically active ingredient previously used to treat said disease or disorder, such as the pharmaceutically active ingredients listed in the Physician's Desk Reference for treating such diseases or disorders).

This invention also provides a method of treating abdominal obesity in a patient in need thereof comprising administering to said patient an effective amount of at least one (e.g., 1, 2 or 3, or 1 or 2, or 1, or 1) compound of formula 1.

This invention also provides a method of treating abdominal obesity in a patient in need thereof comprising administering to said patient an effective amount of a compound of formula 1.

This invention also provides a method of treating abdominal obesity in a patient in need thereof, comprising administering to said patient an effective amount of at least one (e.g., 1, 2 or 3, or 1 or 2, or 1, or 1) compound of formula 1, in combination with at least one (e.g., 1, 2 or 3, or 1 or 2, or 1, or 1) other pharmaceutically active ingredient (such as a pharmaceutically active ingredient previously used to treat said disease or disorder, such as the pharmaceutically active ingredients listed in the Physician's Desk Reference for treating abdominal obesity).

This invention also provides a method of treating abdominal obesity in a patient in need thereof, comprising administering to said patient an effective amount of a compound of formula 1, in combination with at least one (e.g., 1, 2 or 3, or 1 or 2, or 1, or 1) other pharmaceutically active ingredient (such as a pharmaceutically active ingredient previously used to treat said disease or disorder, such as the pharmaceutically active ingredients listed in the Physician's Desk Reference for treating abdominal obesity).

This invention also provides the pharmaceutical compositions described above wherein a pharmaceutically acceptable salt of the compound of formula is used in the composition.

This invention also provides the methods described above wherein a pharmaceutically acceptable salt of the compound of formula is used in the method.

This invention also provides the pharmaceutical compositions described above wherein a solvate of the compound of formula 1 is used.

This invention also provides the pharmaceutical compositions described above wherein a stereoisomer of the compound of formula 1 is used.

This invention also provides pharmaceutical compositions as described above wherein a pharmaceutically acceptable salt of at least one (e.g., 1, 2 or 3, or 1 or 2, or 1, and usually 1) compound of formula 1 is used.

This invention also provides pharmaceutical compositions as described above wherein at least one (e.g., 1, 2 or 3, or 1 or 2, or 1, and usually 1) compound of formula 1 is used.

This invention also provides pharmaceutical compositions described above wherein a compound of formula 1 is used.

This invention also provides methods as described above wherein a solvate of at least one (e.g., 1, 2 or 3, or 1 or 2, or 1, and usually 1) compound of formula 1 is used.

This invention also provides methods as described above wherein a stereoisomer of at least one (e.g., 1, 2 or 3, or 1 or 2, or 1, and usually 1) compound of formula 1 is used.

This invention also provides methods as described above wherein a pharmaceutically acceptable salt of at least one (e.g., 1, 2 or 3, or 1 or 2, or 1, and usually 1) compound of formula 1 is used.

This invention also provides methods as described above wherein at least one (e.g., 1, 2 or 3, or 1 or 2, or 1, and usually 1) compound of formula 1 is used.

This invention also provides methods as described above wherein a compound of formula 1 is used.

### DETAILED DESCRIPTION OF THE INVENTION

As used above, and throughout this disclosure, the following terms, unless otherwise indicated, shall be understood to have the meanings described below.

“At least one” means 1 or more than 1 (e.g., 1, 2 or 3, or 1 or 2, or 1).

“One or more” means 1 or more than 1 (e.g., 1, 2 or 3, or 1 or 2, or 1).

“Patient” includes both human and animals.

“Mammal” means humans and other mammalian animals.

“The metabolic syndrome” is characterized by a group of metabolic risk factors in one patient (e.g., one person). The risk factors include, for example: (a) abdominal obesity (excessive fat tissue in and around the abdomen), (b) atherogenic dyslipidemia (blood fat disorders—high triglycerides, low HDL cholesterol and high LDL cholesterol—that foster plaque buildups in the artery walls), and (c) insulin resistance or glucose intolerance (the body can't properly use insulin or blood sugar).

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain “Lower alkyl” means a group having about 1 to about 6 carbon atoms in the chain which may be straight or branched. The term “substituted alkyl” means that the alkyl group may be substituted by one or more substituents which may be the same or different, each substituent being independently selected from the group consisting of halogen, alkyl, aryl, cycloalkyl, cyano, hydroxy, alkoxy, alkylthio, amino, —NH(alkyl), —NH(cycloalkyl), —N(alkyl)₂, carboxy and —C(O)O-alkyl. Non-limiting examples of suitable alkyl groups include methyl, ethyl, n-propyl, isopropyl and t-butyl.

“Alkylene” means a divalent group obtained by removal of a hydrogen atom from an alkyl group that is defined above. Non-limiting examples of alkylene include methylene, ethylene (i.e., —CH₂CH₂— or —CH(CH₃)—) and propylene (e.g., including —CH₂CH₂CH₂— and —CH(CH₃)CH₂—).

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. The term “substituted alkenyl” means that the alkenyl group may be substituted by one or more substituents which may be the same or different, each substituent being independently selected from the group consisting of halogen, alkyl. aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Alkenylene” means a divalent group obtained by removal of a hydrogen from an alkenyl group that is defined above. Non-limiting examples of alkenylene include —CH═CH—, —C(CH₃)—CH═, and —CH═CHCH₂—.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl and 3-methylbutynyl. The term “substituted alkynyl” means that the alkynyl group may be substituted by one or more substituents which may be the same or different, each substituent being independently selected from the group consisting of alkyl, aryl and cycloalkyl.

“Alkynylene” means a difunctional group obtained by removal of a hydrogen from an alkynyl group that is defined above. Non-limiting examples of alkenylene include —C≡C— and —CH₂C≡C—.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl, indazolyl, and the like, in which there is at least one aromatic ring.

“Aralkyl”, “arylalkyl”, or “-alkylene-aryl” means an aryl-alkyl- group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl- group in which the alkyl and aryl are as previously described. Preferred alkylaryls comprise a lower alkyl group. A non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like, as well as partially saturated species such as, for example, indanyl, tetrahydronaphthyl and the like.

“Halogen” or “halo” means fluorine, chlorine, bromine, or iodine. Preferred are fluorine, chlorine and bromine.

“Ring system substituent” means a substituent attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, acyl, aroyl, halogen, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, —C(═N—CN)—NH₂, —C(═NH)NH₂, —C(═NH)—NH(alkyl), Y₁Y₂N—, Y₁Y₂N-alkyl-, Y₁Y₂NC(O)—, Y₁Y₂NSO₂— and —SO₂NY₁Y₂, wherein Y₁ and Y₂ can be the same or different and are independently selected from the group consisting of hydrogen, alkyl, aryl, cycloalkyl, and aralkyl. “Ring system substituent” may also mean a single moiety which simultaneously replaces two available hydrogens on two adjacent carbon atoms (one H on each carbon) on a ring system. Examples of such moiety are methylenedioxy, ethylenedioxy, —C(CH₃)₂— and the like which form moieties such as, for example:

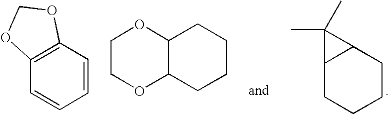

“Heterocyclyl” means a monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Heterocyclyls may be completely saturated, partially unsaturated, or aromatic. Aromatic heterocyclyls are termed “heteroaryl”, as defined above. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include saturated heterocyclyls, for example piperidyl, pyrrolidinyl, piperazinyl, morpholinyl, thiomorpholinyl, thiazolidinyl, 1,4-dioxanyl, tetrahydrofuranyl, tetrahydrothiophenyl, lactams, lactones, and the like. Non-limiting examples of partially unsaturated monocyclic heterocyclyl rings include, for example, thiazolinyl, and the like.

It should be noted that in heteroatom containing ring systems of this invention, there are no hydroxyl groups on carbon atoms adjacent to a N, O or S, as well as there are no N or S groups on carbon adjacent to another heteroatom. Thus, for example, in the ring: 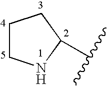
there is no —OH attached directly to carbons marked 2 and 5.

It should also be noted that the compounds of the present invention include tautomers of the compounds of Formula 1.

“Heteroaralkyl” means a heteroaryl-alkyl- group in which the heteroaryl and alkyl are as previously described. Preferred heteroaralkyls contain a lower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl- group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an H—C(O)—, alkyl-C(O)— or cycloalkyl-C(O)—, group in which the various groups are as previously described. The bond to the parent moiety is through the carbonyl. Preferred acyls contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl. Non-limiting examples of suitable groups include benzoyl and 1-naphthoyl.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Alkoxycarbonyl” means an alkyl-O—CO— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

The term “purified”, “in purified form” or “in isolated and purified form” for a compound refers to the physical state of said compound after being isolated from a synthetic process or natural source or combination thereof. Thus, the term “purified”, “in purified form” or “in isolated and purified form” for a compound refers to the physical state of said compound after being obtained from a purification process or processes described herein or well known to the skilled artisan, in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al., Protective Groups in Organic Synthesis (1991), Wiley, New York.

When any variable (e.g., aryl, heterocycle, R⁹, etc.) occurs more than one time in any constituent or in Formula 1, its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. The term “prodrug”, as employed herein, denotes a compound that is a drug precursor which, upon administration to a subject, undergoes chemical conversion by metabolic or chemical processes to yield a compound of Formula I or a salt and/or solvate thereof. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press, both of which are incorporated herein by reference thereto.

“Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

“Effective amount” or “therapeutically effective amount” is meant to describe an amount of compound or a composition of the present invention effective in inhibiting the diseases or conditions noted below, and thus producing the desired therapeutic, ameliorative, inhibitory or preventative effect.

Thus, this invention provides compounds of formula 1: 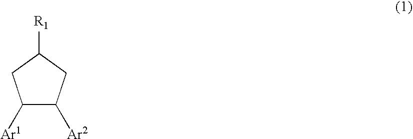
or the pharmaceutically acceptable salts thereof, wherein:

Ar¹ is

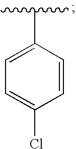

Ar² is

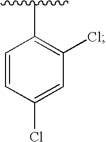

R is —(CH₂)ₘ—X—(CH₂)ₙ—R²;

X is selected from the group consisting of: —NH—, —O—, —C(O)— and —S(O)₂—;

R² is

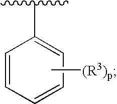

R³ is selected from the group consisting of halo (e.g., F) and —CN;

m is 0 to 4;

n is 0 or 1; and

p is 1, 2, or 3.

As stated above, this invention also provides pharmaceutically acceptable salts of formula 1.

This invention also provides solvates of formula 1.

The compounds of formula 1 include stereoisomers of formula 1. These stereoisomers include, for example:

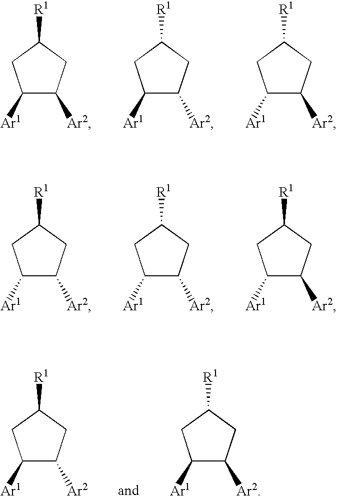

Thus, one embodiment of this invention is directed to compounds of formula 1 having the formula:

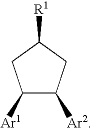

Another embodiment of this invention is directed to compounds of formula 1 having the formula:

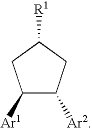

Another embodiment of this invention is directed to compounds of formula 1 having the formula:

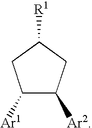

Another embodiment of this invention is directed to compounds of formula 1 having the formula:

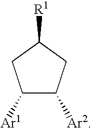

Another embodiment of this invention is directed to compounds of formula 1 having the formula:

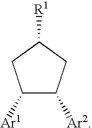

Another embodiment of this invention is directed to compounds of formula 1 having the formula:

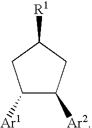

Another embodiment of this invention is directed to compounds of formula 1 having the formula:

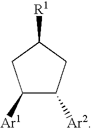

Another embodiment of this invention is directed to compounds of formula 1 having the formula:

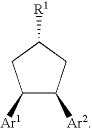

Preferably, the compounds of formula 1 are compounds of the formula:

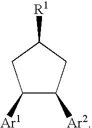

Preferably, X is selected from the group consisting of: —NH— and —O—. Most preferably, X is —NH—.

Preferably R³ is selected from the group consisting of: F and —CN. Most preferably, R³ is —CN.

Preferably, p is 1 or 2. Most preferably, p is 1 when R³ is —CN, and p is 2 when R³ is F.

Thus, in one embodiment R² is selected from the group consisting of:

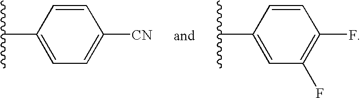

In another embodiment R² is:

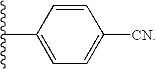

In another embodiment R² is:

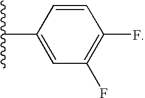

Preferably, m is 1.

In one embodiment n is 0.

In another embodiment n is 1.

Thus, examples of R¹ include, for example:

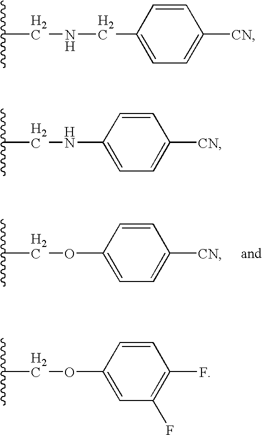

Preferably, R¹ is:

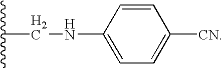

Representative examples of the compounds of formula 1 include, for example:

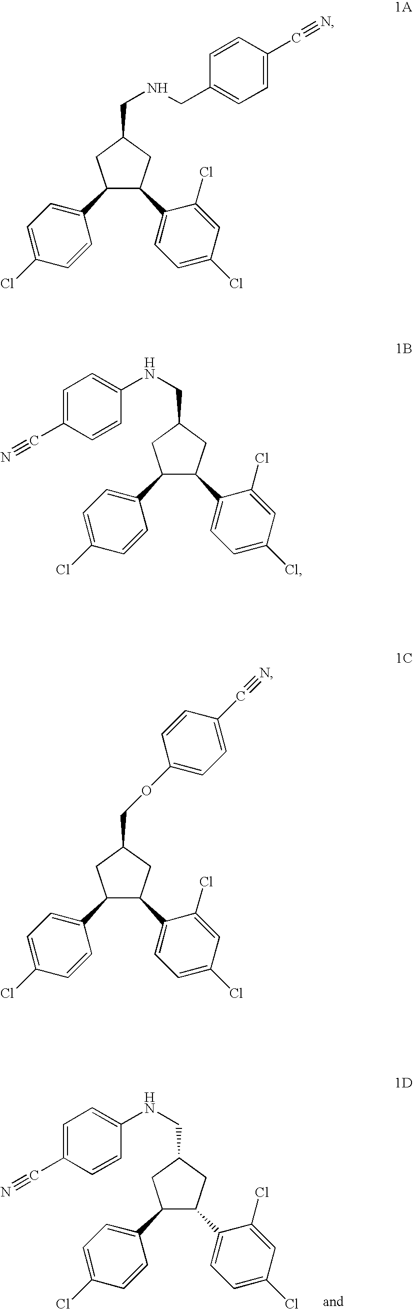

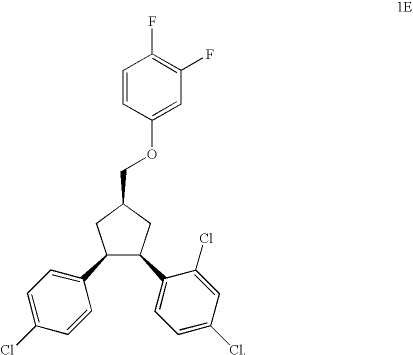

Thus one embodiment of this invention is directed to compound 1A:

Another embodiment of this invention is directed to a pharmaceutically acceptable salt of compound 1A

Another embodiment of this invention is directed to a solvate of compound 1A.

Another embodiment of this invention is directed to the stereoisomers of compound 1A.

Another embodiment of this invention is directed to compound 1B.

Another embodiment of this invention is directed to a pharmaceutically acceptable salt of compound 1B

Another embodiment of this invention is directed to a solvate of compound 1B.

Another embodiment of this invention is directed to the stereoisomers of compound 1B.

Another embodiment of this invention is directed to compound 1C.

Another embodiment of this invention is directed to a pharmaceutically acceptable salt of compound 1C

Another embodiment of this invention is directed to a solvate of compound 1C.

Another embodiment of this invention is directed to the stereoisomers of compound 1C.

Another embodiment of this invention is directed to compound 1D.

Another embodiment of this invention is directed to a pharmaceutically acceptable salt of compound 1D

Another embodiment of this invention is directed to a solvate of compound 1D.

Another embodiment of this invention is directed to the stereoisomers of compound 1D.

Another embodiment of this invention is directed to compound 1E.

Another embodiment of this invention is directed to a pharmaceutically acceptable salt of compound 1E

Another embodiment of this invention is directed to a solvate of compound 1E.

Another embodiment of this invention is directed to the stereoisomers of compound 1E.

Another embodiment of this invention is directed to a pharmaceutical composition comprising a compound of 1A and a pharmaceutically acceptable carrier.

Another embodiment of this invention is directed to a pharmaceutical composition comprising a pharmaceutically acceptable salt of the compound of 1A and a pharmaceutically acceptable carrier.

Another embodiment of this invention is directed to a pharmaceutical composition comprising a solvate of the compound of 1A and a pharmaceutically acceptable carrier.

Another embodiment of this invention is directed to a pharmaceutical composition comprising a stereoisomer of the compound of 1A and a pharmaceutically acceptable carrier.

Another embodiment of this invention is directed to a pharmaceutical composition comprising a compound of 1B and a pharmaceutically acceptable carrier.

Another embodiment of this invention is directed to a pharmaceutical composition comprising a pharmaceutically acceptable salt of the compound of 1B and a pharmaceutically acceptable carrier.

Another embodiment of this invention is directed to a pharmaceutical composition comprising a solvate of the compound of 1B and a pharmaceutically acceptable carrier.

Another embodiment of this invention is directed to a pharmaceutical composition comprising a stereoisomer of the compound of 1B and a pharmaceutically acceptable carrier.

Another embodiment of this invention is directed to a pharmaceutical composition comprising a compound of 1C and a pharmaceutically acceptable carrier.

Another embodiment of this invention is directed to a pharmaceutical composition comprising a pharmaceutically acceptable salt of the compound of 1C and a pharmaceutically acceptable carrier.

Another embodiment of this invention is directed to a pharmaceutical composition comprising a solvate of the compound of 1C and a pharmaceutically acceptable carrier.

Another embodiment of this invention is directed to a pharmaceutical composition comprising a stereoisomer of the compound of 1C and a pharmaceutically acceptable carrier.

Another embodiment of this invention is directed to a pharmaceutical composition comprising a compound of 1D and a pharmaceutically acceptable carrier.

Another embodiment of this invention is directed to a pharmaceutical composition comprising a pharmaceutically acceptable salt of the compound of 1D and a pharmaceutically acceptable carrier.

Another embodiment of this invention is directed to a pharmaceutical composition comprising a solvate of the compound of 1D and a pharmaceutically acceptable carrier.

Another embodiment of this invention is directed to a pharmaceutical composition comprising a stereoisomer of the compound of 1D and a pharmaceutically acceptable carrier.

Another embodiment of this invention is directed to a pharmaceutical composition comprising a compound of 1E and a pharmaceutically acceptable carrier.

Another embodiment of this invention is directed to a pharmaceutical composition comprising a pharmaceutically acceptable salt of the compound of 1E and a pharmaceutically acceptable carrier.

Another embodiment of this invention is directed to a pharmaceutical composition comprising a solvate of the compound of 1E and a pharmaceutically acceptable carrier.

Another embodiment of this invention is directed to a pharmaceutical composition comprising a stereoisomer of the compound of 1E and a pharmaceutically acceptable carrier.

Another embodiment of this invention is directed to anyone of the methods of treatment described herein wherein the compound of formula 1 that is used is a compound of formula 1A.

Another embodiment of this invention is directed to anyone of the methods of treatment described herein wherein the compound of formula 1 that is used is a pharmaceutically acceptable salt of the compound of formula 1A.

Another embodiment of this invention is directed to anyone of the methods of treatment described herein wherein the compound of formula 1 that is used is a solvate of the compound of formula 1A.

Another embodiment of this invention is directed to anyone of the methods of treatment described herein wherein the compound of formula 1 that is used is a stereoisomer of the compound of formula 1A.

Another embodiment of this invention is directed to anyone of the methods of treatment described herein wherein the compound of formula 1 that is used is a compound of formula 1B.

Another embodiment of this invention is directed to anyone of the methods of treatment described herein wherein the compound of formula 1 that is used is a pharmaceutically acceptable salt of the compound of formula 1B.

Another embodiment of this invention is directed to anyone of the methods of treatment described herein wherein the compound of formula 1 that is used is a solvate of the compound of formula 1B.

Another embodiment of this invention is directed to anyone of the methods of treatment described herein wherein the compound of formula 1 that is used is a stereoisomer of the compound of formula 1B.

Another embodiment of this invention is directed to anyone of the methods of treatment described herein wherein the compound of formula 1 that is used is a compound of formula 1C.

Another embodiment of this invention is directed to anyone of the methods of treatment described herein wherein the compound of formula 1 that is used is a pharmaceutically acceptable salt of the compound of formula 1C.

Another embodiment of this invention is directed to anyone of the methods of treatment described herein wherein the compound of formula 1 that is used is a solvate of the compound of formula 1C.

Another embodiment of this invention is directed to anyone of the methods of treatment described herein wherein the compound of formula 1 that is used is a stereoisomer of the compound of formula 1C.

Another embodiment of this invention is directed to anyone of the methods of treatment described herein wherein the compound of formula 1 that is used is a compound of formula 1D.

Another embodiment of this invention is directed to anyone of the methods of treatment described herein wherein the compound of formula 1 that is used is a pharmaceutically acceptable salt of the compound of formula 1D.

Another embodiment of this invention is directed to anyone of the methods of treatment described herein wherein the compound of formula 1 that is used is a solvate of the compound of formula 1D.

Another embodiment of this invention is directed to anyone of the methods of treatment described herein wherein the compound of formula 1 that is used is a stereoisomer of the compound of formula 1D.

Another embodiment of this invention is directed to anyone of the methods of treatment described herein wherein the compound of formula 1 that is used is a compound of formula 1E.

Another embodiment of this invention is directed to anyone of the methods of treatment described herein wherein the compound of formula 1 that is used is a pharmaceutically acceptable salt of the compound of formula 1E.

Another embodiment of this invention is directed to anyone of the methods of treatment described herein wherein the compound of formula 1 that is used is a solvate of the compound of formula 1E.

Another embodiment of this invention is directed to anyone of the methods of treatment described herein wherein the compound of formula 1 that is used is a stereoisomer of the compound of formula 1E.

Another embodiment of this invention is directed to any one of the methods of treatment described herein wherein pharmaceutical composition is used, and wherein said pharmaceutical composition comprises a compound of formula 1A and a pharmaceutically acceptable carrier.

Another embodiment of this invention is directed to any one of the methods of treatment described herein wherein pharmaceutical composition is used, and wherein said pharmaceutical composition comprises a pharmaceutically acceptable salt of a compound of formula 1A and a pharmaceutically acceptable carrier.

Another embodiment of this invention is directed to any one of the methods of treatment described herein wherein pharmaceutical composition is used, and wherein said pharmaceutical composition comprises a solvate of a compound of formula 1A and a pharmaceutically acceptable carrier.

Another embodiment of this invention is directed to any one of the methods of treatment described herein wherein pharmaceutical composition is used, and wherein said pharmaceutical composition comprises a stereoisomer of a compound of formula 1A and a pharmaceutically acceptable carrier.

Another embodiment of this invention is directed to any one of the methods of treatment described herein wherein pharmaceutical composition is used, and wherein said pharmaceutical composition comprises a compound of formula 1B and a pharmaceutically acceptable carrier.

Another embodiment of this invention is directed to any one of the methods of treatment described herein wherein pharmaceutical composition is used, and wherein said pharmaceutical composition comprises a pharmaceutically acceptable salt of a compound of formula 1B and a pharmaceutically acceptable carrier.

Another embodiment of this invention is directed to any one of the methods of treatment described herein wherein pharmaceutical composition is used, and wherein said pharmaceutical composition comprises a solvate of a compound of formula 1B and a pharmaceutically acceptable carrier.

Another embodiment of this invention is directed to any one of the methods of treatment described herein wherein pharmaceutical composition is used, and wherein said pharmaceutical composition comprises a stereoisomer of a compound of formula 1B and a pharmaceutically acceptable carrier.

Another embodiment of this invention is directed to any one of the methods of treatment described herein wherein pharmaceutical composition is used, and wherein said pharmaceutical composition comprises a compound of formula 1C and a pharmaceutically acceptable carrier.

Another embodiment of this invention is directed to any one of the methods of treatment described herein wherein pharmaceutical composition is used, and wherein said pharmaceutical composition comprises a pharmaceutically acceptable salt of a compound of formula 1C and a pharmaceutically acceptable carrier.

Another embodiment of this invention is directed to any one of the methods of treatment described herein wherein pharmaceutical composition is used, and wherein said pharmaceutical composition comprises a solvate of a compound of formula 1C and a pharmaceutically acceptable carrier.

Another embodiment of this invention is directed to any one of the methods of treatment described herein wherein pharmaceutical composition is used, and wherein said pharmaceutical composition comprises a stereoisomer of a compound of formula 1C and a pharmaceutically acceptable carrier.

Another embodiment of this invention is directed to any one of the methods of treatment described herein wherein pharmaceutical composition is used, and wherein said pharmaceutical composition comprises a compound of formula 1D and a pharmaceutically acceptable carrier.

Another embodiment of this invention is directed to any one of the methods of treatment described herein wherein pharmaceutical composition is used, and wherein said pharmaceutical composition comprises a pharmaceutically acceptable salt of a compound of formula 1D and a pharmaceutically acceptable carrier.

Another embodiment of this invention is directed to any one of the methods of treatment described herein wherein pharmaceutical composition is used, and wherein said pharmaceutical composition comprises a solvate of a compound of formula 1D and a pharmaceutically acceptable carrier.

Another embodiment of this invention is directed to any one of the methods of treatment described herein wherein pharmaceutical composition is used, and wherein said pharmaceutical composition comprises a stereoisomer of a compound of formula 1D and a pharmaceutically acceptable carrier.

Another embodiment of this invention is directed to any one of the methods of treatment described herein wherein pharmaceutical composition is used, and wherein said pharmaceutical composition comprises a compound of formula 1E and a pharmaceutically acceptable carrier.

Another embodiment of this invention is directed to any one of the methods of treatment described herein wherein pharmaceutical composition is used, and wherein said pharmaceutical composition comprises a pharmaceutically acceptable salt of a compound of formula 1E and a pharmaceutically acceptable carrier.

Another embodiment of this invention is directed to any one of the methods of treatment described herein wherein pharmaceutical composition is used, and wherein said pharmaceutical composition comprises a solvate of a compound of formula 1E and a pharmaceutically acceptable carrier.

Another embodiment of this invention is directed to any one of the methods of treatment described herein wherein pharmaceutical composition is used, and wherein said pharmaceutical composition comprises a stereoisomer of a compound of formula 1E and a pharmaceutically acceptable carrier.

The compounds of Formula 1 can form salts which are also within the scope of this invention. Reference to a compound of Formula 1 herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula 1 contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., nontoxic, physiologically acceptable) salts are preferred, although other salts are also useful. Salts of the compounds of the Formula 1 may be formed, for example, by reacting a compound of Formula 1 with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

One or more compounds of the invention may also exist as, or optionally converted to, a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

Compounds of Formula 1, and salts, solvates, and prodrugs thereof, may exist in their tautomeric form (for example, as an amide or imino ether). All such tautomeric forms are contemplated herein as part of the present invention.

All stereoisomers (for example, geometric isomers, optical isomers and the like) of the present compounds (including those of the salts, solvates, and prodrugs of the compounds as well as the salts and solvates of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 3-Cl-phenyl and 4-Cl-phenyl). Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “prodrug” and the like, is intended to equally apply to the salt, solvate, and prodrug of enantiomers, stereoisomers, rotamers, tautomers, positional isomers, racemates or prod rugs of the inventive compounds.

Polymorphic forms of the compounds of formula 1, and of the salts, solvates and prodrugs of the compounds of formula 1, are intended to be included in the present invention.

The compounds of formula 1, or pharmaceutically acceptable salts or solvates, thereof according to the invention have pharmacological properties; in particular, the compounds of formula 1 can be selective CB₁ antagonists. The term “selective” means that the compounds of formula 1 bind to the CB₁ receptor more strongly than to other cannabinoid receptors.

Certain compounds useful in the therapeutic compositions or combinations of the invention may have at least one asymmetric carbon atom and therefore all isomers, including enantiomers, diastereomers, stereoisomers, rotamers, tautomers and racemates of the compounds of Formula 1 (where they exist) are contemplated as being part of this invention. The invention includes single enantiomers and mixtures of enatiomers in both pure form and in admixture, including racemic mixtures. Isomers can be prepared using conventional techniques, either by reacting optically pure or optically enriched starting materials or by separating isomers of a compound of the formulae 1. Isomers may also include diastereomers and geometric isomers, e.g., when a double bond is present.

Those skilled in the art will appreciate that for some of the compounds of the formulae 1, one isomer may show greater pharmacological activity than other isomers.

The compounds of Formula 1 of the present invention, or pharmaceutically acceptable salts, or solvates, thereof are useful in treating diseases or conditions mediated by or involving a cannabinoid receptor (e.g., a CB1 receptor). The diseases or conditions include, for example: the metabolic syndrome (e.g., abdominal obesity, atherogenic dyslipidemia, insulin resistance, and glucose intolerance), neuroinflammatory disorders, addictive behavior, diseases of the central nervous system, cardiovascular disorders, respiratory disorders, gastrointestinal disorders, insulin sensitivity, diabetes mellitus, hypertriglyceridemia, eating disorders, alcoholism, inflammation, psychiatric disorders, migraine, nicotine dependence, Parkinson's disease, psychosis, schizophrenia, sleep disorders, attention deficit hyperactivity disorder, male sexual dysfunction, premature ejaculation, premenstrual syndrome, seizure, epilepsy and convulsion, non-insulin dependent diabetes, dementia, major depressive disorder, bulimia nervosa, drug dependence, septic shock, cognitive disorder, endocrine disorders, eczema, emesis, allergy, glaucoma, hemorrhagic shock, hypertension, angina, thrombosis, atherosclerosis, restenosis, hypertension, acute coronary syndrome, angina pectoris, arrhythmia, heart failure, cerebral ischemia, stroke, myocardial infarction, glomerulonephritis, thrombotic and thromboembolytic stroke, peripheral vascular diseases, neurodegenerative disease, osteoporosis, pulmonary disease, autoimmune disease, hypotension, arthropathy, cancer, demyelinating diseases, Alzheimer's disease, hypoactive sexual desire disorder, bipolar disorder, hyperlipidemia, hypertension, narcotic dependence, Huntington's chorea, pain, multiple sclerosis, anxiety disorder, bone disorders, Paget's disease, rheumatoid arthritis, ulcerative colitis, irritable bowel syndrome, and inflammatory bowel diseases.

The metabolic syndrome is characterized by a group of metabolic risk factors in one patient (e.g., one person). The risk factors include, for example: (a) abdominal obesity (excessive fat tissue in and around the abdomen), (b) atherogenic dyslipidemia (blood fat disorders—high triglycerides, low HDL cholesterol and high LDL cholesterol—that foster plaque buildups in the artery walls), and (c) insulin resistance or glucose intolerance (the body can't properly use insulin or blood sugar).

The term “pharmaceutical composition” is also intended to encompass both the bulk composition and individual dosage units comprised of more than one (e.g., two) pharmaceutically active agents such as, for example, a compound of the present invention and an additional agent selected from the lists of the additional agents described herein, along with any pharmaceutically inactive excipients. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit such as tablets, pills and the like. Similarly, the herein-described method of treating a patient by administering a pharmaceutical composition of the present invention is also intended to encompass the administration of the afore-said bulk composition and individual dosage units.

The compounds of formula 1, or pharmaceutically acceptable salts, solvates, or esters thereof, can be administered in any suitable form, e.g., alone, or in combination with a pharmaceutically acceptable carrier, excipient or diluent in a pharmaceutical composition, according to standard pharmaceutical practice. The compounds of formula 1, or pharmaceutically acceptable salts, solvates, or esters thereof, can be administered orally or parenterally, including intravenous, intramuscular, interperitoneal, subcutaneous, rectal, or topical routes of administration.

Pharmaceutical compositions comprising at least one compound of formula 1, or a pharmaceutically acceptable salt or solvate thereof can be in a form suitable for oral administration, e.g., as tablets, troches, capsules, lozenges, aqueous or oily suspensions, dispersible powders or granules, emulsions, syrups, or elixirs. Oral compositions may be prepared by any conventional pharmaceutical method, and may also contain sweetening agents, flavoring agents, coloring agents, and preserving agents.

The amount of compound of formula 1, or a pharmaceutically acceptable salt, or solvate thereof, administered to a patient can be determined by a physician based on the age, weight, and response of the patient, as well as by the severity of the condition treated. For example, the amount of compound of formula 1, or a pharmaceutically acceptable salt or solvate thereof, administered to the patient can range from about 0.1 mg/kg body weight per day to about 60 mg/kg/d, preferably about 0.5 mg/kg/d to about 40 mg/kg/d.

The compounds of formula 1 can be administered in combination (e.g., sequentially or concurrently) with at least one (e.g., 1, 2 or 3, or 1 or 2, or 1, and usually 1) other pharmaceutically active ingredient (i.e., other therapeutic agent). Examples of these other therapeutic agents that can be used in combination with the compounds of formula 1 include, but are not limited to: cholesterol lowering agents, substituted azetidinone or substituted β-lactam sterol absorption inhibitors, sterol absorption inhibitors, cholesterol biosynthesis inhibitors, lipid-lowering compounds, bile acid sequestrants (insoluble anion exchange resins), nicotinic acid (niacin) and/or derivatives thereof, AcylCoA: Cholesterol O-acyltransferase (“ACAT”) Inhibitors, Cholesteryl Ester Transfer Protein (“CETP”) Inhibitors, probucol or derivatives thereof, low-density lipoprotein (LDL) receptor activators, fish oil, natural water soluble fibers, plant sterols, plant stanols and/or fatty acid esters of plant stanols, antioxidants, monocyte and macrophage inhibitors, hormone replacement agents and compositions (e.g., androgens, estrogens, progestins, their pharmaceutically acceptable salts and derivatives thereof), obesity control medications, blood modifiers, cardiovascular agents, and antidiabetic medications for reducing blood glucose levels in a human.

The compounds of formula 1, or pharmaceutically acceptable salts, or solvates, thereof, can also be administered in combination with other therapeutic agents. For example one or more (e.g., 1, 2 or 3, or 1 or 2, or 1, and usually 1) compounds of formula 1, or pharmaceutically acceptable salts, or solvates, thereof, can be administered with one or more (e.g., 1, 2 or 3, or 1 or 2, or 1, or 1) additional cholesterol lowering agents.

A non-limiting list of cholesterol lowering agents useful in the present invention include HMG CoA reductase inhibitor compounds such as lovastatin (for example MEVACOR® which is available from Merck & Co.), simvastatin (for example ZOCOR® which is available from Merck & Co.), pravastatin (for example PRAVACHOL® which is available from Bristol Meyers Squibb), atorvastatin calcium (for example LIPITOR® which is available from Pfizer, Inc.), fluvastatin, cerivastatin, CI-981, rivastatin (sodium 7-(4-fluorophenyl)-2,6-diisopropyl-5-methoxymethylpyridin-3-yl)-3,5-dihydroxy-6-heptanoate), rosuvastatin calcium (CRESTOR® from AstraZeneca Pharmaceuticals), pitavastatin (such as NK-104 of Negma Kowa of Japan); HMG CoA synthetase inhibitors, for example L-659,699 ((E,E)-11-[3′R-(hydroxy-methyl)-4′-oxo-2′R-oxetanyl]-3,5,7R-trimethyl-2,4-undecadienoic acid); squalene synthesis inhibitors, for example squalestatin 1; squalene epoxidase inhibitors, for example, NB-598 ((E)-N-ethyl-N-(6,6-dimethyl-2-hepten-4-ynyl)-3-[(3,3′-bithiophen-5-yl)methoxy]benzene-methanamine hydrochloride); sterol (e.g., cholesterol) biosynthesis inhibitors such as DMP-565; nicotinic acid derivatives (e.g., compounds comprising a pyridine-3-carboxylate structure or a pyrazine-2-carboxylate structure, including acid forms, salts, esters, zwitterions and tautomers) such as niceritrol, nicofuranose and acipimox (5-methylpyrazine-2-carboxylic acid 4-oxide); clofibrate; gemfibrazol; bile acid sequestrants such as cholestyramine (a styrene-divinylbenzene copolymer containing quaternary ammonium cationic groups capable of binding bile acids, such as QUESTRAN® or QUESTRAN LIGHT® cholestyramine which are available from Bristol-Myers Squibb), colestipol (a copolymer of diethylenetriamine and 1-chloro-2,3-epoxypropane, such as COLESTID® tablets which are available from Pharmacia), colesevelam hydrochloride (such as WelChol® Tablets (poly(allylamine hydrochloride) cross-linked with epichlorohydrin and alkylated with 1-bromodecane and (6-bromohexyl)-trimethylammonium bromide) which are available from Sankyo), water soluble derivatives such as 3,3-ioene, N-(cycloalkyl) alkylamines and poliglusam, insoluble quaternized polystyrenes, saponins and mixtures thereof; inorganic cholesterol sequestrants such as bismuth salicylate plus montmorillonite clay, aluminum hydroxide and calcium carbonate antacids; ileal bile acid transport (“IBAT”) inhibitors (or apical sodium co-dependent bile acid transport (“ASBT”) inhibitors) such as benzothiepines, for example the therapeutic compounds comprising a 2,3,4,5-tetrahydro-1-benzothiepine 1,1-dioxide structure such as are disclosed in PCT Patent Application WO 00/38727 which is incorporated herein by reference; AcylCoA: Cholesterol O-acyltransferase (“ACAT”) Inhibitors such as avasimibe ([[2,4,6-tris(1-methylethyl)phenyl]acetyl]sulfamic acid, 2,6-bis(1-methylethylphenyl ester, formerly known as CI-1011), HL-004, lecimibide (DuP-128) and CL-277082 (N-(2,4-difluorophenyl)-N-[[4-(2,2-dimethylpropyl)phenyl]methyl]-N-heptylurea), and the compounds described in P. Chang et al., “Current, New and Future Treatments in Dyslipidaemia and Atherosclerosis”, Drugs 2000 July; 60(1); 55-93, which is incorporated by reference herein; Cholesteryl Ester Transfer Protein (“CETP”) Inhibitors such as those disclosed in PCT Patent Application No. WO 00/38721 and U.S. Pat. No. 6,147,090, which are incorporated herein by reference; NAR agonists (such as, for example, Niacin and pharmaceutical compositions and combinations comprising Niacin); microsomal triglyceride transfer protein (“MTTP”) antagonists; and peroxisome proliferating receptor (“PPR”) agonists (such as, for example, gemfibrozil); probucol or derivatives thereof, such as AGI-1067 and other derivatives disclosed in U.S. Pat. Nos. 6,121,319 and 6,147,250, herein incorporated by reference; low-density lipoprotein (LDL) receptor activators such as HOE402, an imidazolidinyl-pyrimidine derivative that directly stimulates LDL receptor activity, described in M. Huettinger et al., “Hypolipidemic activity of HOE-402 is Mediated by Stimulation of the LDL Receptor Pathway”, Arterioscler. Thromb. 1993; 13:1005-12, herein incorporated by reference; fish oils containing Omega 3 fatty acids (3-PUFA); natural water soluble fibers, such as psyllium, guar, oat and pectin; plant stanols and/or fatty acid esters of plant stanols, such as sitostanol ester used in BENECOL® margarine; nicotinic acid receptor agonists (e.g., agonists of the HM74 and HM74A receptor which receptor is described in US 2004/0142377, US 2005/0004178, US 2005/0154029, U.S. Pat. No. 6,902,902, WO 2004/071378, WO 2004/071394, WO 01/77320, US 2003/0139343, WO 01/94385, WO 2004/083388, US 2004/254224, US 2004/0254224, US 2003/0109673 and WO 98/56820) for example those described in WO 2004/033431, WO 2005/011677, WO 2005/051937, US 2005/0187280, US 2005/0187263, WO 2005/077950, WO 2005/016867, and WO 2005/016870; and the substituted azetidinone or substituted β-lactam sterol absorption inhibitors discussed in detail below.

As used herein, “sterol absorption inhibitor” means a compound capable of inhibiting the absorption of one or more sterols, including but not limited to cholesterol, phytosterols (such as sitosterol, campesterol, stigmasterol and avenosterol), 5α-stanols (such as cholestanol, 5α-campestanol, 5α-sitostanol), and/or mixtures thereof, when administered in a therapeutically effective (sterol and/or 5α-stanol absorption inhibiting) amount to a mammal or human.

### Substituted Azetidinones Useful in Combination with the Compounds of the Present Invention

In one embodiment, substituted azetidinones useful in the compositions, therapeutic combinations and methods of the present invention are represented by Formula (A) below: 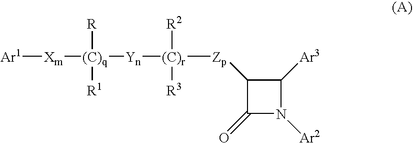
or pharmaceutically acceptable salts, solvates, or esters of the compounds of Formula (A), wherein, in Formula (A) above:

Ar¹ and Ar² are independently selected from the group consisting of aryl and R⁴-substituted aryl;

Ar³ is aryl or R⁵-substituted aryl;

X, Y and Z are independently selected from the group consisting of —CH₂—, —CH(lower alkyl)- and —C(lower alkyl)₂-;

R and R² are independently selected from the group consisting of —OR, —OC(O)R⁶, —OC(O)OR⁹ and —OC(O)NR⁶R⁷;

R¹ and R³ are independently selected from the group consisting of hydrogen, lower alkyl and aryl;

q is 0 or 1; r is 0 or 1; m, n and p are independently selected from 0, 1, 2, 3 or 4; provided that at least one of q and r is 1, and the sum of m, n, p, q and r is 1, 2, 3, 4, 5 or 6; and provided that when p is 0 and r is 1, the sum of m, q and n is 1, 2, 3, 4 or 5;

R⁴ is 1-5 substituents independently selected from the group consisting of lower alkyl, —OR⁶, —OC(O)R⁶, —OC(O)OR⁹, —O(CH₂)₁₋₅OR⁶, —OC(O)NR⁶R⁷, —NR⁶R⁷, —NR⁶C(O)R⁷, —NR⁶C(O)OR⁹, —NR⁶C(O)NR⁷R⁸, —NR⁶SO₂R⁹, —C(O)OR⁶, —C(O)NR⁶R⁷, —C(O)R⁶, —S(O)₂NR⁶R⁷, S(O)₀₋₂R⁹, —O(CH₂)₁₋₁₀—C(O)OR⁶, —O(CH₂)₁₋₁₀CONR⁶R⁷, -(lower alkylene)COOR⁶, —CH—CH═C(O)OR⁶, —CF₃, —CN, —NO₂ and halogen;

R⁵ is 1-5 substituents independently selected from the group consisting of —OR⁶, —OC(O)R⁶, —OC(O)OR⁹, —O(CH₂)₁₋₅OR⁶, —OC(O)NR⁶R⁷, —NR⁶R₇—NR⁶C(O)R⁷, —NR⁶C(O)OR⁹, —NR⁶C(O)NR⁷R₈, —NR⁶S(O)₂R⁹, —C(O)OR⁶, —C(O)NR⁶R⁷, —C(O)R⁶, —SO₂NR⁶R⁷, S(O)₀₋₂R⁹, —O(CH₂)₁₋₁₀—C(O)OR⁶, —O(CH₂)₁₋₁₀C(O)NR⁶R⁷, -(lower alkylene)C(O)OR⁶ and —CH═CH—C(O)OR⁶;

R⁶, R⁷ and R⁸ are independently selected from the group consisting of hydrogen, lower alkyl, aryl and aryl-substituted lower alkyl; and

R⁹ is lower alkyl, aryl or aryl-substituted lower alkyl.

Preferably, R⁴ is 1-3 independently selected substituents, and R⁵ is preferably 1-3 independently selected substituents.

Certain compounds useful in the therapeutic compositions or combinations of the invention may have at least one asymmetrical carbon atom and therefore all isomers, including enantiomers, diastereomers, stereoisomers, rotamers, tautomers and racemates of the compounds of Formula A-M (where they exist) are contemplated as being part of this invention. The invention includes d and l isomers in both pure form and in admixture, including racemic mixtures. Isomers can be prepared using conventional techniques, either by reacting optically pure or optically enriched starting materials or by separating isomers of a compound of the Formulae A-M. Isomers may also include geometric isomers, e.g., when a double bond is present.

Those skilled in the art will appreciate that for some of the compounds of the Formulae A-M, one isomer may show greater pharmacological activity than other isomers.

Preferred compounds of Formula (A) are those in which Ar¹ is phenyl or R⁴-substituted phenyl, more preferably (4-R⁴)-substituted phenyl. Ar² is preferably phenyl or R⁴-substituted phenyl, more preferably (4-R⁴)-substituted phenyl. Ar³ is preferably R⁵-substituted phenyl, more preferably (4-R⁵)-substituted phenyl. When Ar¹ is (4-R⁴)-substituted phenyl, R⁴ is preferably a halogen. When Ar² and Ar³ are R⁴- and R⁵-substituted phenyl, respectively, R⁴ is preferably halogen or —OR⁶ and R⁵ is preferably —OR⁶, wherein R⁶ is lower alkyl or hydrogen. Especially preferred are compounds wherein each of Ar¹ and Ar² is 4-fluorophenyl and Ar³ is 4-hydroxyphenyl or 4-methoxyphenyl.

X, Y and Z are each preferably —CH₂—. R¹ and R³ are each preferably hydrogen. R and R² are preferably —OR⁶ wherein R⁶ is hydrogen, or a group readily metabolizable to a hydroxyl (such as —OC(O)R⁶, —OC(O)OR⁹ and —OC(O)NR⁶R⁷ defined above).

The sum of m, n, p, q and r is preferably 2, 3 or 4, more preferably 3. Preferred are compounds of Formula (A) wherein m, n and r are each zero, q is 1 and p is 2.

Also preferred are compounds of Formula (A) in which p, q and n are each zero, r is 1 and m is 2 or 3. More preferred are compounds wherein m, n and r are each zero, q is 1, p is 2, Z is —CH₂— and R is —OR⁶, especially when R⁶ is hydrogen.

Also more preferred are compounds of Formula (A) wherein p, q and n are each zero, r is 1, m is 2, X is —CH₂— and R² is —OR⁶, especially when R⁶ is hydrogen.

Another group of preferred compounds of Formula (A) is that in which Ar¹ is phenyl or R⁴-substituted phenyl, Ar² is phenyl or R⁴-substituted phenyl and Ar³ is R⁵-substituted phenyl. Also preferred are compounds in which Ar¹ is phenyl or R⁴-substituted phenyl, Ar² is phenyl or R⁴-substituted phenyl, Ar³ is R⁵-substituted phenyl, and the sum of m, n, p, q and r is 2, 3 or 4, more preferably 3. More preferred are compounds wherein Ar¹ is phenyl or R⁴-substituted phenyl, Ar² is phenyl or R⁴-substituted phenyl, Ar³ is R⁵-substituted phenyl, and wherein m, n and r are each zero, q is 1 and p is 2, or wherein p, q and n are each zero, r is 1 and m is 2 or 3.

Substituted Azetidinones of Formula (B)

In a preferred embodiment, a substituted azetidinone of Formula (A) useful in the compositions, therapeutic combinations and methods of the present invention is represented by Formula (B) (ezetimibe) below: 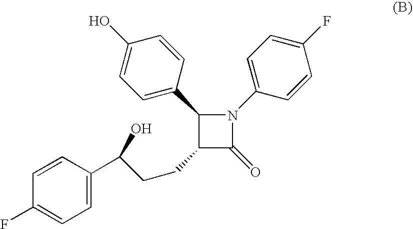
or pharmaceutically acceptable salts, solvates, or esters of the compound of Formula (B). The compound of Formula (B) can be in anhydrous or hydrated form. A product containing ezetimibe compound is commercially available as ZETIA® ezetimibe formulation from MSP Pharmaceuticals.

Compounds of Formula (A) can be prepared by a variety of methods well known to those skilled in the art, for example such as are disclosed in U.S. Pat. Nos. 5,631,365, 5,767,115, 5,846,966, 6,207,822, 6,627,757, 6,093,812, 5,306,817, 5,561,227, 5,688,785, and 5,688,787, each of which is incorporated herein by reference.

Substituted Azetidinones of Formula (C)

Alternative substituted azetidinones useful in the compositions, therapeutic combinations and methods of the present invention are represented by Formula (C) below: 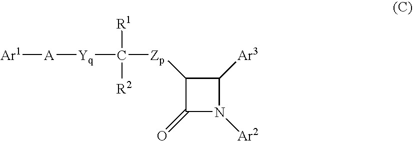
or a pharmaceutically acceptable salt thereof or a solvate thereof, or an ester thereof, wherein, in Formula (C) above:

Ar¹ is R³-substituted aryl;

Ar² is R⁴-substituted aryl;

Ar³ is R⁵-substituted aryl;

Y and Z are independently selected from the group consisting of —CH₂—, —CH(lower alkyl and —C(lower alkyl)₂—;

A is selected from —O—, —S—, —S(O)— or —S(O)₂—;

R¹ is selected from the group consisting of —OR⁶, —OC(O)R⁶, —OC(O)OR⁹ and —OC(O)NR⁶R⁷;

R² is selected from the group consisting of hydrogen, lower alkyl and aryl; or R¹ and R² together are ═O;

q is 1, 2 or 3;

p is 0, 1, 2, 3 or 4;

R⁵ is 1-3 substituents independently selected from the group consisting of —OR⁶, —OC(O)R⁶, —OC(O)OR⁹, —O(CH₂)₁₋₅OR⁹, —OC(O)NR⁶R⁷, —NR⁶R⁷, —NR⁶C(O)R⁷, —NR⁶C(O)OR⁹, —NR⁶C(O)NR⁷R⁸, —NR⁶S(O)₂-lower alkyl, —NR⁶S(O)₂-aryl, —C(O)NR⁶R⁷, —COR⁶, —SO₂NR⁶R⁷, —S(O)₀₋₂-alkyl, S(O)₀₋₂-aryl, —O(CH₂)₁₋₁₀—C(O)OR⁶, —O(CH₂)₁₋₁₀C(O)NR⁶R⁷, -halogeno, m-halogeno, o-lower alkyl, m-lower alkyl, -(lower alkylene)-C(O)OR⁶, and —CH═CH—C(O)OR⁶;

R³ and R⁴ are independently 1-3 substituents independently selected from the group consisting of R⁵, hydrogen, p-lower alkyl, aryl, —NO₂, —CF₃ and p-halogeno;

R⁶, R⁷ and R⁸ are independently selected from the group consisting of hydrogen, lower alkyl, aryl and aryl-substituted lower alkyl; and R⁹ is lower alkyl, aryl or aryl-substituted lower alkyl.

Methods for making compounds of Formula (C) are well known to those skilled in the art. Non-limiting examples of suitable methods are disclosed in U.S. Pat. No. 5,688,990, which is incorporated herein by reference.

Substituted Azetidinones of Formula (D)

In another embodiment, substituted azetidinones useful in the compositions, therapeutic combinations and methods of the present invention are represented by Formula (D): 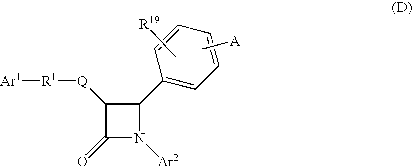
or a pharmaceutically acceptable salt thereof or a solvate thereof, or an ester thereof, wherein, in Formula (D) above:

A is selected from the group consisting of R²-substituted heterocycloalkyl, R²-substituted heteroaryl, R²-substituted benzo-fused heterocycloalkyl, and R²-substituted benzo-fused heteroaryl;

Ar¹ is aryl or R³-substituted aryl;

Ar² is aryl or R⁴-substituted aryl;

Q is a bond or, with the 3-position ring carbon of the azetidinone, forms the spiro group 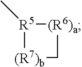
and

R¹ is selected from the group consisting of:
—(CH₂)_{q}—, wherein q is 2-6, provided that when Q forms a spiro ring, q can also be zero or 1;
—(CH₂)ₑ-G-(CH₂)ᵣ—, wherein G is —O—, —C(O)—, phenylene, —NR⁸— or
—S(O)₀₋₂—, e is 0-5 and r is 0-5, provided that the sum of e and r is 1-6;
—(C₂-C₆ alkenylene)-; and
—(CH₂)_{f}-V-(CH₂)_{g}—, wherein V is C₃-C₆ cycloalkylene, f is 1-5 and g is 0-5, provided that the sum of f and g is 1-6;

R⁵ is selected from: 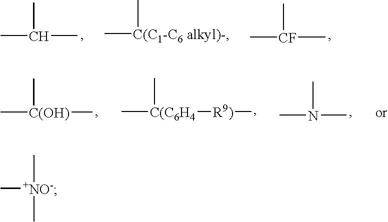
R⁶ and R⁷ are independently selected from the group consisting of —CH₂—, —CH(C₁-C₆ alkyl)-, —C(di-(C₁-C₆) alkyl), —CH═CH— and —C(C₁-C₆ alkyl)-CH—; or R⁵ together with an adjacent R⁶, or R⁵ together with an adjacent R⁷, form a —CH═CH— or a —CH═C(C₁-C₆ alkyl)- group;

a and b are independently 0, 1, 2 or 3, provided both are not zero; provided that when R⁶ is —CH═CH— or —C(C₁-C₆ alkyl)═CH—, a is 1; provided that when R⁷ is —CH═CH— or —C(C₁-C₆ alkyl)═CH—, b is 1, provided that when a is 2 or 3, the R⁶'s can be the same or different; and provided that when b is 2 or 3, the R⁷'s can be the same or different,

and when Q is a bond, R¹ also can be selected from: 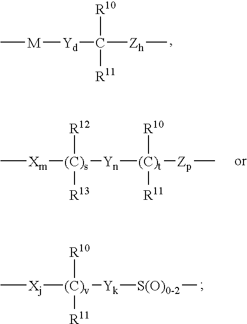
where M is —O—, —S—, —S(O)— or —S(O)₂—;

X, Y and Z are independently selected from the group consisting of —CH₂—, —CH(C₁-C₆ alkyl)- and —C(di-(C₁-C₆) alkyl);

R¹⁰ and R¹² are independently selected from the group consisting of —OR¹⁴, —OC(O)R¹⁴, —OC(O)OR¹⁶ and —OC(O)NR¹⁴R¹⁵;

R¹¹ and R¹³ are independently selected from the group consisting of hydrogen, (C₁-C₆)alkyl and aryl; or R¹⁰ and R¹¹ together are ═O, or R¹² and R¹³ together are ═O;

d is 1, 2 or 3;

h is 0, 1, 2, 3 or 4;

s is 0 or 1; t is 0 or 1; m, n and p are independently 0-4; provided that at least one of s and t is 1, and the sum of m, n, p, s and t is 1-6; provided that when p is 0 and t is 1, the sum of m, s and n is 1-5; and provided that when p is 0 and s is 1, the sum of m, t and n is 1-5;

v is 0 or 1;

j and k are independently 1-5, provided that the sum of j, k and v is 1-5;

R² is 1-3 substituents on the ring carbon atoms selected from the group consisting of hydrogen, (C₁-C₁₀)alkyl, (C₂-C₁₀)alkenyl, (C₂-C₁₀)alkynyl, (C₃-C₆)cycloalkyl, (C₃-C₆)cycloalkenyl, R¹⁷-substituted aryl, R¹⁷-substituted benzyl, R¹⁷-substituted benzyloxy, R¹⁷-substituted aryloxy, halogeno, —NR¹⁴R¹⁵, NR¹⁴R¹⁵(C₁-C₆ alkylene)-, NR¹⁴R¹⁵C(O)(C₁-C₆ alkylene)-, —NHC(O)R¹⁶, OH, C₁-C₆ alkoxy, —OC(O)R¹⁶, —C(O)R¹⁴, hydroxy(C₁-C₆)alkyl, (C₁-C₆)alkoxy(C₁-C₆)alkyl, NO₂, —S(O)₀₋₂R¹⁶, —S(O)₂NR¹⁴R¹⁵ and —(C₁-C₆ alkylene)C(O)OR¹⁴; when R² is a substituent on a heterocycloalkyl ring, R² is as defined, or R² is ═O or 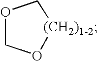
and, where R² is a substituent on a substitutable ring nitrogen, R² is hydrogen, (C₀-C₆)alkyl, aryl, (C₁-C₆)alkoxy, aryloxy, (C₁-C₆)alkylcarbonyl, arylcarbonyl, hydroxy, —(CH₂)₁₋₆CONR¹⁸R¹⁸, or 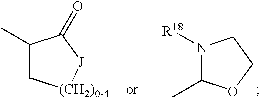
wherein J is —O—, —NH—, —NR¹⁸— or —CH₂—;

R³ and R⁴ are independently selected from the group consisting of 1-3 substituents independently selected from the group consisting of (C₁-C₆)alkyl, —OR¹⁴, —OC(O)R¹⁴, —OC(O)OR¹⁶, —O(CH₂)₁₋₅OR¹⁴, —OC(O)NR¹⁴R¹⁵, —NR¹⁴R¹⁵, —NR¹⁴C(O)R¹⁵, —NR¹⁴C(O)R¹⁶, —NR¹⁴C(O)NR¹⁵R¹⁹, —NR¹⁴S(O)₂R¹⁶, —C(O)OR¹⁴, —C(O)NR¹⁴R¹⁵, —C(O)R¹⁴, —S(O)₂NR¹⁴R¹⁵, S(O)₀₋₂R¹⁶, —O(CH₂)₁₋₁₀—C(O)OR¹⁴, —O(CH₂)₁₋₁₀C(O)NR¹⁴R¹⁵, —(C₁-C₆ alkylene)-C(O)OR¹⁴, —CH═CH—C(O)OR¹⁴, —CF₃, —CN, —NO₂ and halogen;

R⁸ is hydrogen, (C₁-C₆)alkyl, aryl (C₁-C₆)alkyl, —C(O)R¹⁴ or —C(O)R¹⁴;

R⁹ and R¹⁷ are independently 1-3 groups independently selected from the group consisting of hydrogen, (C₁-C₆)alkyl, (C₁-C₆)alkoxy, —C(O)OH, NO₂, —NR¹⁴R¹⁵, OH and halogeno;

R¹⁴ and R¹⁵ are independently selected from the group consisting of hydrogen, (C₁-C₆)alkyl, aryl and aryl-substituted (C₁-C₆)alkyl;

R¹⁶ is (C₁-C₆)alkyl, aryl or R¹⁷-substituted aryl;

R¹⁸ is hydrogen or (C₁-C₆)alkyl; and

R¹⁹ is hydrogen, hydroxy or (C₁-C₆)alkoxy.

Methods for making compounds of Formula (D) are well known to those skilled in the art. Non-limiting examples of suitable methods are disclosed in U.S. Pat. No. 5,656,624, which is incorporated herein by reference.

Substituted Azetidinones of Formula (E)

In another embodiment, substituted azetidinones useful in the compositions, therapeutic combinations and methods of the present invention are represented by Formula (E): 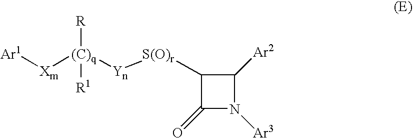
or a pharmaceutically acceptable salt thereof or a solvate thereof, or an ester thereof, wherein, in Formula (E) above:

Ar¹ is aryl, R¹⁰-substituted aryl or heteroaryl;

Ar² is aryl or R⁴-substituted aryl;

Ar³ is aryl or R⁵-substituted aryl;

X and Y are independently selected from the group consisting of —CH₂—, —CH(lower alkyl)- and —C(lower alkyl)₂-;

R is —OR⁶, —OC(O)R⁶, —OC(O)OR⁹ or —OC(O)NR⁶R⁷; R¹ is hydrogen, lower alkyl or aryl; or R and R¹ together are ═O;

q is 0 or 1;

r is 0, 1 or 2;

m and n are independently 0, 1, 2, 3, 4 or 5; provided that the sum of m, n and q is 1, 2, 3, 4 or 5;

R⁴ is 1-5 substituents independently selected from the group consisting of lower alkyl, —OR⁶, —OC(O)R⁶, —OC(O)OR⁹, —O(CH₂)₁₋₅OR⁶, —OC(O)NR⁶R⁷, —NR⁶R⁷, —NR⁶C(O)R⁷, —NR⁶C(O)OR⁹, —NR⁶C(O)NR⁷R⁸, —NR⁶S(O)₂R⁹, —C(O)OR⁶, —C(O)NR⁶R⁷, —C(O)R⁶; —S(O)₂NR⁶R⁷, S(O)₀₋₂R⁹, —O(CH₂)₁₋₁₀—C(O)OR⁶, —O(CH₂)₁₋₁₀CO(O)NR⁶R⁷, -(lower alkylene)C(O)OR⁶ and —CH═CH—C(O)OR⁶;

R⁵ is 1-5 substituents independently selected from the group consisting of —OR⁶, —OC(O)R⁶, —OC(O)OR⁹, —O(CH₂)₁₋₅OR⁶, —OC(O)NR⁶R⁷, —NR⁶R⁷, —NR⁶C(O)R⁷, —NR⁶C(O)OR⁹, —NR⁶C(O)NR⁷R⁸, —NR⁶S(O)₂R⁹, —C(O)OR⁶, —C(O)NR⁶R⁷, —C(O)R⁶, —S(O)₂NR⁶R⁷, S(O)₀₋₂R⁹, —O(CH₂)₁₋₁₀—C(O)OR⁶, —O(CH₂)₁₋₁₀C(O(NR⁶R⁷, —CF₃, —CN, —NO₂, halogen, -(lower alkylene)C(O)OR⁶ and —CH═CH—C(O)OR⁶;

R⁶, R⁷ and R⁸ are independently selected from the group consisting of hydrogen, lower alkyl, aryl and aryl-substituted lower alkyl;

R⁹ is lower alkyl, aryl or aryl-substituted lower alkyl; and

R¹⁰ is 1-5 substituents independently selected from the group consisting of lower alkyl, —OR⁶, —OC(O)R⁶, —OC(O)OR⁹, —O(CH₂)₁₋₅OR⁶, —OC(O)NR⁶R⁷, —NR⁶R⁷, —NR⁶C(O)R⁷, —NR⁶C(O)OR⁹, —NR⁶C(O)NR⁷R⁸, —NR⁶S(O)₂R⁹, —C(O)OR⁶, —C(O)NR⁶R⁷, C(O)R⁶, —S(O)₂NR⁶R⁷, —S(O)₀₋₂R⁹, —O(CH₂)₁₋₁₀—O(O)OR⁶, —O(CH₂)₁₋₁₀C(O)NR⁶R⁷, —CF₃, —CN, —NO₂ and halogen.

Methods for making compounds of Formula (E) are well known to those skilled in the art. Non-limiting examples of suitable methods are disclosed in U.S. Pat. No. 5,624,920, which is incorporated herein by reference.

In another embodiment, substituted azetidinones useful in the compositions, therapeutic combinations and methods of the present invention are represented by Formula (F): 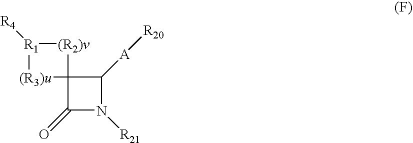
or a pharmaceutically acceptable salt thereof or a solvate thereof, or an ester thereof wherein:

R¹ is:

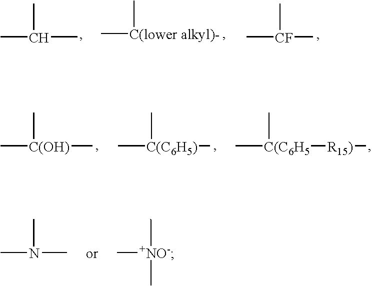

R² and R³ are independently selected from the group consisting of: —CH₂—, —CH(lower alkyl)-, —C(lower alkyl)₂—, —CH═CH— and —C(lower alkyl)═CH—; or

R¹ together with an adjacent R², or R¹ together with an adjacent R³, form a —CH═CH— or a —CH═C(lower alkyl)- group;

u and v are independently 0, 1, 2 or 3, provided both are not zero; provided that when R² is —CH═CH— or —C(lower alkyl)═CH—, v is 1; provided that when R³ is —CH═CH— or —C(lower alkyl)═CH—, u is 1; provided that when v is 2 or 3, each R² can be the same or different; and provided that when u is 2 or 3, each R³ can be the same or different;

R⁴ is selected from B—(CH₂)ₘC(O)—, wherein m is 0, 1, 2, 3, 4 or 5;

B—(CH₂)_{q}—, wherein q is 0, 1, 2, 3, 4, 5 or 6; B—(CH₂)ₑ-Z-(CH₂)_{c}, wherein Z is —O—, —C(O)—, phenylene, —N(R⁸)— or —S(O)₀₋₂—, e is 0, 1, 2, 3, 4 or 5 and r is 0, 1, 2, 3, 4 or 5, provided that the sum of e and r is 0, 1, 2, 3, 4, 5 or 6; B—(C₂-C₆ alkenylene)-; B—(C₄-C₆ alkadienylene)-; B—(CH₂)ₜ-Z-(C₂-C₆ alkenylene)-, wherein Z is as defined above, and wherein t is 0, 1, 2 or 3, provided that the sum of t and the number of carbon atoms in the alkenylene chain is 2, 3, 4, 5 or 6; B—(CH₂)_{f}—V—(CH₂)_{g}—, wherein V is C₃-C₆ cycloalkylene, f is 1, 2, 3, 4 or 5 and g is 0, 1, 2, 3, 4 or 5, provided that the sum of f and g is 1, 2, 3, 4, 5 or 6; B—(CH₂)ₜ—V—(C₂-C₆ alkenylene)- or B—(C₂-C₆ alkenylene)-V—(CH₂)ₜ—, wherein V and t are as defined above, provided that the sum of t and the number of carbon atoms in the alkenylene chain is 2, 3, 4, 5 or 6;

B—(CH₂)ₐ-Z-(CH₂)_{b}—V—(CH₂)_{d}—, wherein Z and V are as defined above and a, b and d are independently 0, 1, 2, 3, 4, 5 or 6, provided that the sum of a, b and d is 0, 1, 2, 3, 4, 5 or 6; or T-(CH₂)ₛ—, wherein T is a C₃-C₆ cycloalkyl and s is 0, 1, 2, 3, 4, 5 or 6; or

R¹ and R⁴ together form the group

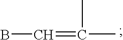

B is selected from indanyl, indenyl, naphthyl, tetrahydronaphthyl, heteroaryl or W-substituted heteroaryl, wherein heteroaryl is selected from the group consisting of pyrrolyl, pyridinyl, pyrimidinyl, pyrazinyl, triazinyl, imidazolyl, thiazolyl, pyrazolyl, thienyl, oxazolyl and furanyl, and for nitrogen-containing heteroaryls, the N-oxides thereof, or

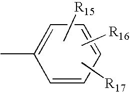

W is 1 to 3 substituents independently selected from the group consisting of lower alkyl, hydroxy lower alkyl, lower alkoxy, alkoxyalkyl, alkoxyalkoxy, alkoxycarbonylalkoxy, (lower alkoxyimino)-lower alkyl, lower alkanedioyl, lower alkyl lower alkanedioyl, allyloxy, —CF₃, —OCF₃, benzyl, R⁷-benzyl, benzyloxy, R⁷-benzyloxy, phenoxy, R⁷-phenoxy, dioxolanyl, NO₂, —N(R⁸)(R⁹), N(R⁸)(R⁹)-lower alkylene-, N(R⁸)(R⁹)-lower alkylenyloxy-, OH, halogeno, —CN, —N₃, —NHC(O)OR¹⁰, —NHC(O)R¹⁰, R¹¹(O)₂SNH—, (R¹¹(O)₂S)₂N—, —S(O)₂NH₂, —S(O)₀₋₂R⁸, tert-butyldimethyl-silyloxymethyl, —C(O)R¹², —C(O)OR¹⁹, —C(O)N(R⁸)(R⁹), —CH═CHC(O)R¹², -lower alkylene-C(O)R¹², R¹⁰C(O)(lower alkylenyloxy)-, N(R⁸)(R⁹)C(O)(lower alkylenyloxy)- and 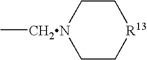
for substitution on ring carbon atoms, and the substituents on the substituted heteroaryl ring nitrogen atoms, when present, are selected from the group consisting of lower alkyl, lower alkoxy, —C(O)OR¹⁰, —C(O)R¹⁰), OH, N(R⁸)(R⁹)-lower alkylene-, N(R⁸)(R⁹)-lower alkylenyloxy-, —S(O)₂NH₂ and 2-(trimethylsilyl)-ethoxymethyl;

R⁷ is 1-3 groups independently selected from the group consisting of lower alkyl, lower alkoxy, —C(O)OH, NO₂, —N(R⁸)(R⁹), OH, and halogeno;

R⁸ and R⁹ are independently selected from H or lower alkyl;

R¹⁰ is selected from lower alkyl, phenyl, R⁷-phenyl, benzyl or R⁷-benzyl;

R¹¹ is selected from OH, lower alkyl, phenyl, benzyl, R⁷-phenyl or R⁷-benzyl;

R¹² is selected from H, OH, alkoxy, phenoxy, benzyloxy, 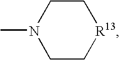
—N(R⁸)(R⁹), lower alkyl, phenyl or R⁷-phenyl;

R¹³ is selected from —O—, —CH₂—, —NH—, —N(lower alkyl) or —NC(O)R¹⁹;

R¹⁵, R¹⁶ and R¹⁷ are independently selected from the group consisting of H and the groups defined for W; or R¹⁵ is hydrogen and R¹⁶ and R¹⁷, together with adjacent carbon atoms to which they are attached, form a dioxolanyl ring;

R¹⁹ is H, lower alkyl, phenyl or phenyl lower alkyl; and

R²⁰ and R²¹ are independently selected from the group consisting of phenyl, W-substituted phenyl, naphthyl, W-substituted naphthyl, indanyl, indenyl, tetrahydronaphthyl, benzodioxolyl, heteroaryl, W-substituted heteroaryl, benzo-fused heteroaryl, W-substituted benzo-fused heteroaryl and cyclopropyl, wherein heteroaryl is as defined above.

Methods for making compounds of Formula (F) are well known to those skilled in the art. Non-limiting examples of suitable methods are disclosed in U.S. Pat. No. 5,698,548, which is incorporated herein by reference.

In another embodiment, substituted azetidinones useful in the compositions, therapeutic combinations and methods of the present invention are represented by by Formula (G), i.e., Formulas (GA) and (GB): 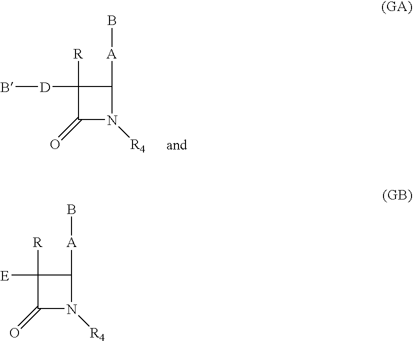
or a pharmaceutically acceptable salt, solvate, or ester thereof, wherein:

A is —CH═CH—, —C≡C— or —(CH₂)ₚ— wherein p is 0, 1 or 2;

B is

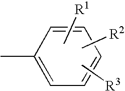

B′ is

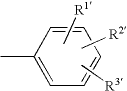

D is —(CH₂)ₘC(O)— or —(CH₂)_{q}— wherein m is 1, 2, 3 or 4 and q is 2, 3 or 4;

E is C₁₀ to C₂₀ alkyl or —C(O)—(C₉ to C₁₉)-alkyl, wherein the alkyl is straight or branched, saturated or containing one or more double bonds;

R is hydrogen, C₁-C₁₅ alkyl, straight or branched, saturated or containing one or more double bonds, or B—(CH₂)ᵣ—, wherein r is 0, 1, 2, or 3;

R¹, R², R₃, R^{1′}, R^{2′}, and R^{3′} are independently selected from the group consisting of hydrogen, lower alkyl, tower alkoxy, carboxy, NO₂, NH₂, OH, halogeno, lower alkylamino, dilower alkylamino, —NHC(O)OR⁵, R⁶(O)₂SNH— and —S(O)₂NH₂;

R⁴ is 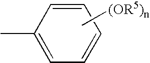
wherein n is 0, 1, 2 or 3;

R⁵ is lower alkyl; and

R⁶ is OH, lower alkyl, phenyl, benzyl or substituted phenyl wherein the substituents are 1-3 groups independently selected from the group consisting of lower alkyl, lower alkoxy, carboxy, NO₂, NH₂, OH, halogeno, lower alkylamino and dilower alkylamino; or a pharmaceutically acceptable salt, solvate, or ester thereof.

Sterol Absorption Inhibitors of Formula (H)

In another embodiment, sterol absorption inhibitors useful in the compositions and methods of the present invention are represented by Formula (H): 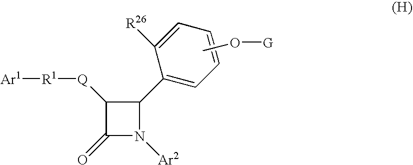
or a pharmaceutically acceptable salt, solvate, or ester thereof, wherein, in Formula (H) above,

R²⁶ is H or OG¹;

G and G¹ are independently selected from the group consisting of 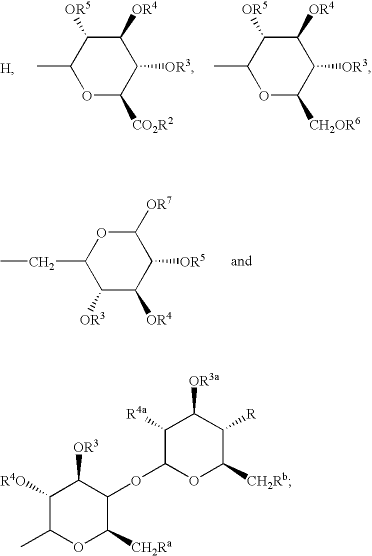
provided that when R²⁶ is H or OH, G is not H;

R, R^{a} and R^{b} are independently selected from the group consisting of H, —OH, halogeno, —NH₂, azido, (C₁-C₆)alkoxy(C₁-C₆)-alkoxy or —W—R³⁰;

W is independently selected from the group consisting of —NH—C(O)—, —O—C(O)—, —O—C(O)—N(R³¹)—, —NH—C(O)—N(R³¹)— and —O—C(S)—N(R³¹)—;

R² and R⁶ are independently selected from the group consisting of H, (C₁-C₆)alkyl, aryl and aryl(C₁-C₆)alkyl;

R³, R⁴, R⁵, R⁷, R^{3a} and R^{4a} are independently selected from the group consisting of H, (C₁-C₆)alkyl, aryl(C₁-C₆)alkyl, —C(O)(C₁-C₆)alkyl and —C(O)aryl;

R³⁰ is selected from the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C₇)cycloalkyl and R³²-substituted-(C₃-C₇)cycloalkyl(C₁-C₆)alkyl;

R³¹ is selected from the group consisting of H and (C₁-C₄)alkyl;

T is selected from the group consisting of phenyl, furyl, thienyl, pyrrolyl, oxazolyl, isoxazolyl, thiazolyl, isothiazolyl, benzothiazolyl, thiadiazolyl, pyrazolyl, imidazolyl and pyridyl;

R³² is independently selected from 1-3 substituents independently selected from the group consisting of halogeno, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N((C₁-C₄)alkyl)₂, —C(O)—(C₁-C₄)alkyl, —C(O)—(C₁-C₄)alkoxy and pyrrolidinylcarbonyl; or

R³² is a covalent bond and R³¹ the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;

Ar¹ is aryl or R¹⁰-substituted aryl;

Ar² is aryl or R¹¹-substituted aryl;

Q is a bond or, with the 3-position ring carbon of the azetidinone, forms the spiro group 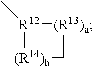
and

R¹ is selected from the group consisting of
—(CH₂)_{q}—, wherein q is 2-6, provided that when Q forms a spiro ring, q can also be zero or 1;
—(CH₂)ₑ-E-(CH₂)ᵣ—, wherein E is —O—, —C(O)—, phenylene, —NR²²— or —S(O)₀₋₂—, e is 0-5 and r is 0-5, provided that the sum of e and r is 1-6;
—(C₂-C₆)alkenylene-; and
—(CH₂)_{f}—V—(CH₂)_{g}—, wherein V is C₃-C₆ cycloalkylene, f is 1-5 and g is 0-5, provided that the sum of f and g is 1-6;

R¹² is:

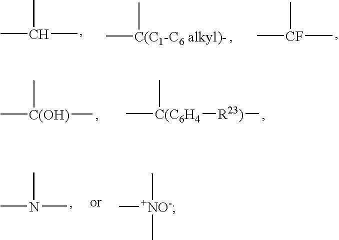

R¹³ and R¹⁴ are independently selected from the group consisting of

—CH₂—, —CH((C₁-C₆)alkyl)-, —C((C₁-C₆) alkyl)₂, —CH═CH— and —C((C₁-C₆) alkyl)═CH—; or

R¹² together with an adjacent R¹³, or R¹² together with an adjacent R¹⁴, form a —CH═CH— or a —CH—C(C₁-C₆ alkyl)- group;

a and b are independently 0, 1, 2 or 3, provided both are not zero;
provided that when R¹³ is —CH═CH— or —C(C₁-C₆ alkyl)═CH—, a is 1;
provided that when R¹⁴ is —CH═CH— or —C(C₁-C₆ alkyl)═CH—, b is 1;
provided that when a is 2 or 3, each R¹³ can be the same or different; and
provided that when b is 2 or 3, each R¹⁴ can be the same or different;
and when Q is a bond, R¹ also can be:

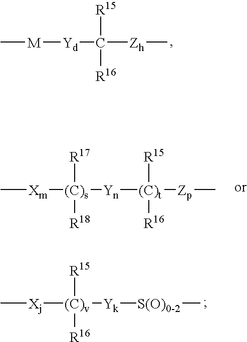

M is —O—, —S—, —S(O)— or —S(O)₂—;

X, Y and Z are independently selected from the group consisting of —CH₂—, —CH(C₁-C₆)alkyl- and —C((C₁-C₆)alkyl)₂;

R¹⁰ and R¹¹ are independently selected from the group consisting of 1-3 substituents independently selected from the group consisting of (C₁-C₆)alkyl, —OR¹⁹, —OC(O)R¹⁹, —OC(O)R²¹, —(OCH₂)₁₋₅OR¹⁹, —OC(O)NR¹⁹R²⁰, —NR¹⁹R²⁰, —NR¹⁹C(O)R²⁰, —NR¹⁹C(O)OR²¹, —NR¹⁹C(O)NR²⁰R²⁵, —NR¹⁹S(O)₂R²¹, —C(O)OR¹⁹, —C(O)NR¹⁹R²⁰, —C(O)R¹⁹, —S(O)₂NR¹⁹R²⁰, S(O)₀₋₂R²¹, —O(CH₂)₁₋₁₀—C(O)OR¹⁹, —O(CH₂)₁₋₁₀C(O)NR¹⁹R²⁰, —C₁-C₆ alkylene)-C(O)OR¹⁹, —CH═CH—C(O)OR¹⁹, —CF₃, —CN, —NO₂ and halogen;

R¹⁵ and R¹⁷ are independently selected from the group consisting of —OR¹⁹, —OC(O)R¹⁹, —OC(O)OR²¹ and —OC(O)NR¹⁹R²⁰;

R¹⁶ and R¹⁸ are independently selected from the group consisting of H, (C₁-C₆)alkyl and aryl; or R¹⁵ and R¹⁶ together are ═O, or R¹⁷ and R¹⁸ together are ═O;

d is 1, 2 or 3;

h is 0, 1, 2, 3 or 4;

s is 0 or 1; t is 0 or 1; m, n and p are independently 0-4;

provided that at least one of s and t is 1, and the sum of m, n, p, s and t is 1-6;

provided that when p is 0 and t is 1, the sum of m, s and n is 1-5; and provided that when p is 0 and s is 1, the sum of m, t and n is 1-5;

v is 0 or 1;

j and k are independently 1-5, provided that the sum of j, k and v is 1-5;

and when Q is a bond and R¹ is 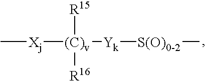
Ar¹ can also be pyridyl, isoxazolyl, furanyl, pyrrolyl, thienyl, imidazolyl, pyrazolyl, thiazolyl, pyrazinyl, pyrimidinyl or pyridazinyl;

R¹⁹ and R²⁰ are independently selected from the group consisting of H, (C₁-C₆)alkyl, aryl and aryl-substituted (C₁-C₆)alkyl;

R²¹ is (C₁-C₆)alkyl, aryl or R²⁴-substituted aryl;

R²² is H, (C₁-C₆)alkyl, aryl (C₁-C₆)alkyl, —C(O)R¹⁹ or —C(O)OR¹⁹;

R²³ and R²⁴ are independently 1-3 groups independently selected from the group consisting of H, (C₁-C₆)alkyl, (C₁-C₆)alkoxy, —C(O)OH, NO₂, —NR¹⁹R²⁰, —OH and halogeno; and

R²⁵ is H, —OH or (C₁-C₆)alkoxy.

Methods for making compounds of Formula (H) are well known to those skilled in the art. Non-limiting examples of suitable methods are disclosed in U.S. Pat. No. 5,756,470, which is incorporated herein by reference.

In another embodiment, substituted azetidinones useful in the compositions and methods of the present invention are represented by Formula (J) below: 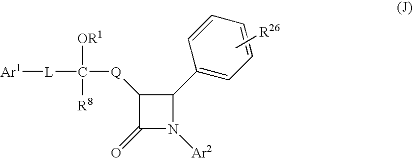
or a pharmaceutically acceptable salt, solvate, or ester thereof wherein in Formula (J):

R¹ is selected from the group consisting of H, G, G¹, G², —SO₃H and —PO₃H;

G is selected from the group consisting of: H, 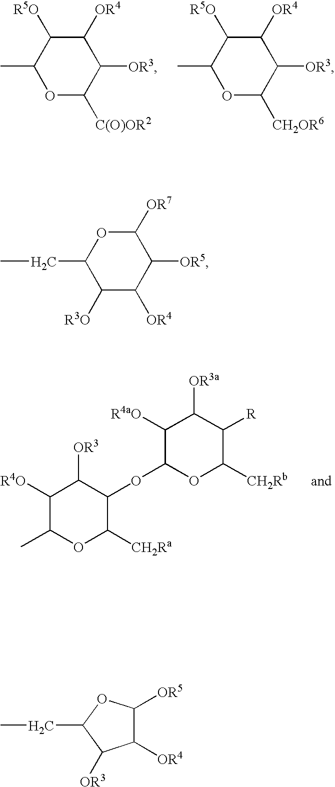
(sugar derivatives)

wherein R, R^{a} and R^{b} are each independently selected from the group consisting of H, —OH, halogen, —NH₂, azido, (C₁-C₆)alkoxy(C₁-C₆)alkoxy or —W—R³⁰;

W is independently selected from the group consisting of —NH—C(O)—, —O—C(O)—, —O—C(O)—N(R³¹)—, —NH—C(O)—N(R³¹)— and —O—C(S)—N(R³¹)—;

R² and R⁶ are each independently selected from the group consisting of H, (C₁-C₆)alkyl, acetyl, aryl and aryl(C₁-C₆)alkyl;

R³, R⁴, R⁵, R⁷, R^{3a} and R^{4a} are each independently selected from the group consisting of H, (C₁-C₆)alkyl, acetyl, aryl(C₁-C₆)alkyl, —C(O)(C₁-C₆)alkyl and —C(O)aryl;

R³⁰ is independently selected from the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C₇)cycloalkyl and R³²-substituted-(C₃-C₇)cycloalkyl(C₁-C₆)alkyl;

R³¹ is independently selected from the group consisting of H and (C₁-C₄)alkyl;

T is independently selected from the group consisting of phenyl, furyl, thienyl, pyrrolyl, oxazolyl, isoxazolyl, thiazolyl, isothiazolyl, benzothiazolyl, thiadiazolyl, pyrazolyl, imidazolyl and pyridyl;

R³² is independently selected from 1-3 substituents which are each independently selected from the group consisting of H, halogen, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N(C₁-C₄)alkyl)₂, —C(O)—(C₁-C₄)alkyl, —C(O)—(C₁-C₄)alkoxy and pyrrolidinylcarbonyl; or

R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;

G¹ is represented by the structure: 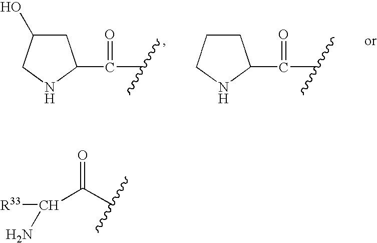
wherein R³³ is independently selected from the group consisting of unsubstituted alkyl, R³⁴-substituted alkyl, (R³⁵)(R³⁶)alkyl-,

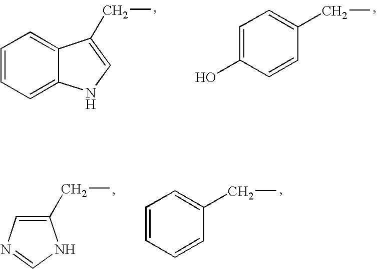

R³⁴ is one to three substituents, each R³⁴ being independently selected from the group consisting of HO(O)C—, HO—, HS—, (CH₃)S—, H₂N—, (NH₂)(NH)C(NH)—, (NH₂)C(O)— and HO(O)CCH(NH₃⁺)CH₂SS—;

R³⁵ is independently selected from the group consisting of H and NH₂—;

R³⁶ is independently selected from the group consisting of H, unsubstituted alkyl, R³⁴-substituted alkyl, unsubstituted cycloalkyl and R³⁴-substituted cycloalkyl;

G² is represented by the structure:

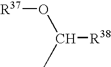

wherein R³⁷ and R³⁸ are each independently selected from the group consisting of (C₁-C₆)alkyl and aryl;

R²⁶ is one to five substituents, each R²⁶ being independently selected from the group consisting of:
a) H;
b) —OH;
c) —OCH₃;
d) fluorine;
e) chlorine;
f) —O-G;
g) —O-G¹;
h) —O-G²;
i) —SO₃H; and
j) —PO₃H;
provided that when R¹ is H, R²⁶ is not H, —OH, —OCH₃ or —O-G;

Ar¹ is aryl, R¹⁰-substituted aryl, heteroaryl or R¹⁰-substituted heteroaryl;

Ar² is aryl, R¹¹-substituted aryl, heteroaryl or R¹¹-substituted heteroaryl;

L is selected from the group consisting of:
a) a covalent bond;
b) —(CH₂)_{q}—, wherein q is 1-6;
c) —(CH₂)ₑ-E-(CH₂)ₜ, wherein E is —O—, —C(O)—, phenylene, —NR²²— or —S(O)₀₋₂—, e is 0-5 and r is 0-5, provided that the sum of e and r is 1-6;
d) —(C₂-C₆)alkenylene-;
e) —(CH₂)_{f}—V—(CH₂)_{g}—, wherein V is C₃-C₆cycloalkylene, f is 1-5 and g is 0-5, provided that the sum of f and g is 1-6; and
f) 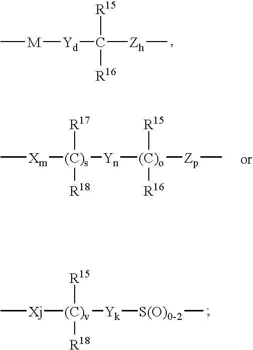
   wherein M is —O—, —S—, —S(O)— or —S(O)₂—;

X, Y and Z are each independently selected from the group consisting of —CH₂—, —CH(C₁-C₆)alkyl- and —C((C₁-C₆)alkyl)₂—;

R⁸ is selected from the group consisting of H and alkyl;

R¹⁰ and R¹¹ are each independently selected from the group consisting of 1-3 substituents which are each independently selected from the group consisting of (C₁-C₆)alkyl, —OR¹⁹, —OC(O)R¹⁹, —OC(O)OR²¹, —O(CH₂)₁₋₅OR¹⁹, —OC(O)NR¹⁹R²⁰, —NR¹⁹R²⁰, —NR¹⁹C(O)R²⁰, —NR¹⁹C(O)OR²¹, —NR¹⁹C(O)NR²⁰R²⁵, —NR¹⁹S(O)₂R²¹, —C(O)OR¹⁹, —C(O)NR¹⁹R²⁰, —C(O)R¹⁹, —S(O)₂NR¹⁹R²⁰, —S(O)₀₋₂R²¹, —O(CH₂)₁₋₁₀—C(O)OR¹⁹, —O(CH₂)₁₋₁₀C(O)NR¹⁹R²⁰, —(C₁-C₆ alkylene)-C(O)OR¹⁹, —CH═CH—C(O)OR¹⁹, —CF₃, —CN, —NO₂ and halogen;

R¹⁵ and R¹⁷ are each independently selected from the group consisting of —OR¹⁹, —OC(O)R¹⁹, —OC(O)OR²¹, —OC(O)NR¹⁹R²⁰;

R¹⁶ and R¹⁸ are each independently selected from the group consisting of H, (C₁-C₆)alkyl and aryl; or

R¹⁵ and R¹⁶ together are ═O, or R¹⁷ and R¹⁸ together are ═O;

d is 1, 2 or 3;

h is 0, 1, 2, 3 or 4;

s is 0 or 1;

t is 0 or 1;

m, n and p are each independently selected from 0-4;

provided that at least one of s and t is 1, and the sum of m, n, p, s and t is 1-6; provided that when p is 0 and t is 1, the sum of m, n and p is 1-5; and provided that when p is 0 and s is 1, the sum of m, t and n is 1-5;

v is 0 or 1;

j and k are each independently 1-5, provided that the sum of j, k and v is 1-5;

Q is a bond, —(CH₂)_{q}—, wherein q is 1-6, or, with the 3-position ring carbon of the azetidinone, forms the spiro group 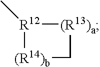
wherein R¹² is

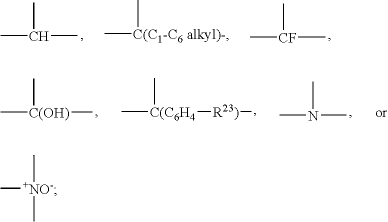

R¹³ and R¹⁴ are each independently selected from the group consisting of —CH₂—, —CH(C₁-C₆ alkyl)-, —C((C₁-C₆)alkyl)₂, —CH═CH— and —C(C₁-C₆ alkyl)═CH—; or R¹² together with an adjacent R¹³, or R¹² together with an adjacent R¹⁴, form a —CH═CH— or a —CH═C(C₁-C₆ alkyl)- group;

a and b are each independently 0, 1, 2 or 3, provided both are not zero; provided that when R¹³ is —CH═CH— or —C(C₁-C₆ alkyl)═CH—, a is 1; provided that when R¹⁴ is —CH═CH— or —C(C₁-C₆ alkyl)═CH—, b is 1; provided that when a is 2 or 3, each R¹³ can be the same or different; and provided that when b is 2 or 3, each R¹⁴ can be the same or different;

and when Q is a bond and L is 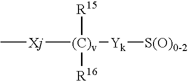
then Ar¹ can also be pyridyl, isoxazolyl, furanyl, pyrrolyl, thienyl, imidazolyl, pyrazolyl, thiazolyl, pyrazinyl, pyrimidinyl or pyridazinyl;

R¹⁹ and R²⁰ are each independently selected from the group consisting of H, (C₁-C₆)alkyl, aryl and aryl-substituted (C₁-C₆)alkyl;

R²¹ is (C₁-C₆)alkyl, aryl or R²⁴-substituted aryl;

R²² is H, (C₁-C₆)alkyl, aryl (C₁-C₆)alkyl, —C(O)R¹⁹ or —C(O)OR¹⁹;

R²³ and R²⁴ are each independently selected from the group consisting of 1-3 substituents which are each independently selected from the group consisting of H, (C₁-C₆)alkyl, (C₁-C₆)alkoxy, —C(O)OH, NO₂, —NR¹⁹R²⁰, —OH and halogen; and

R²⁵ is H, —OH or (C₁-C₆)alkoxy.

Examples of compounds of Formula (J) which are useful in the methods and combinations of the present invention and methods for making such compounds are disclosed in U.S. patent application Ser. No. 10/166,942, filed Jun. 11, 2002, incorporated herein by reference.

An example of a useful substituted azetidinone is one represented by the Formula (K):

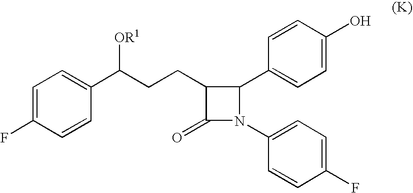

wherein R¹ is defined as above (see, for example, Formula (A)).

A more preferred compound is one represented by Formula (L):

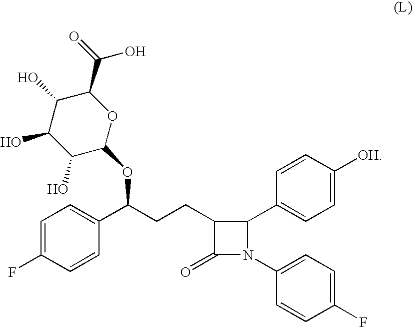

Another useful compound is represented by Formula (M):

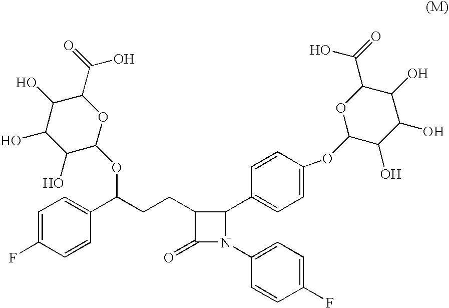

Other useful substituted azetidinone compounds include N-sulfonyl-2-azetidinones such as are disclosed in U.S. Pat. No. 4,983,597, ethyl 4-(2-oxoazetidin-4-yl)phenoxy-alkanoates such as are disclosed in Ram et al., Indian J. Chem. Sect. B. 29B, 12 (1990), p. 1134-7, diphenyl azetidinones and derivatives disclosed in U.S. Patent Publication Nos. 2002/0039774, 2002/0128252, 2002/0128253 and 2002/0137689, 2004/063929, WO 2002/066464, U.S. Pat. Nos. 6,498,156 and 6,703,386, each of which is incorporated by reference herein.

Other sterol absorption inhibitors useful in the compositions, therapeutic combinations and methods of the present invention are described in WO 2004/005247, WO 2004/000803, WO 2004/000804, WO 2004/000805, WO 0250027, U.S. published application 2002/0137689, and the compounds described in L. Kværnø et al., Angew. Chem. Int. Ed., 2004, vol. 43, pp. 4653-4656, all of which are incorporated herein by reference. An illustrative compound of Kværnø et al. is:

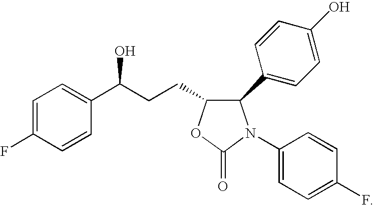

The daily dose of the sterol absorption inhibitor(s) administered to the subject can range from about 0.1 to about 1000 mg per day, preferably about 0.25 to about 50 mg/day, and more preferably about 10 mg per day, given in a single dose or 2-4 divided doses. The exact dose, however, is determined by the attending clinician and is dependent on the potency of the compound administered, the age, weight, condition and response of the patient.

For administration of pharmaceutically acceptable salts of the above compounds, the weights indicated above refer to the weight of the acid equivalent or the base equivalent of the therapeutic compound derived from the salt.

In another embodiment of the present invention, the compositions or therapeutic combinations described above comprise one or more (e.g., 1, 2 or 3, or 1 or 2, or 1, and usually 1) selective CB₁ receptor antagonist compounds of formula 1 in combination with one or more (e.g., 1, 2 or 3, or 1 or 2, or 1, and usually 1) cholesterol biosynthesis inhibitors and/or lipid-lowering compounds discussed below.

Generally, a total daily dosage of cholesterol biosynthesis inhibitor(s) can range from about 0.1 to about 160 mg per day, and preferably about 0.2 to about 80 mg/day in single or 2-3 divided doses.

In another alternative embodiment, the compositions, therapeutic combinations or methods of the present invention can comprise at least one compound of formula 1, or pharmaceutically acceptable salts or solvates thereof, and one or more (e.g., 1, 2 or 3, or 1 or 2, or 1, and usually 1) bile acid sequestrants (insoluble anion exchange resins), co-administered with or in combination with the compound of formula 1, or a pharmaceutically acceptable salt or solvate thereof, and a substituted azetidinone or a substituted β-lactam discussed above.

Bile acid sequestrants bind bile acids in the intestine, interrupting the enterohepatic circulation of bile acids and causing an increase in the faecal excretion of steroids. Use of bile acid sequestrants is desirable because of their non-systemic mode of action. Bile acid sequestrants can lower intrahepatic cholesterol and promote the synthesis of apo B/E (LDL) receptors that bind LDL from plasma to further reduce cholesterol levels in the blood.

Generally, a total daily dosage of bile acid sequestrant(s) can range from about 1 to about 50 grams per day, and preferably about 2 to about 16 grams per day in single or 2-4 divided doses.

In an alternative embodiment, the compositions or treatments of the present invention can comprise at least one (e.g., 1, 2 or 3, or 1 or 2, or 1, and usually 1) compound of Formula 1, or pharmaceutically acceptable salts or solvates thereof, and one or more (e.g., 1, 2 or 3, or 1 or 2, or 1, and usually 1) IBAT inhibitors. The IBAT inhibitors can inhibit bile acid transport to reduce LDL cholesterol levels. Generally, a total daily dosage of IBAT inhibitor(s) can range from about 0.01 to about 1000 mg/day, and preferably about 0.1 to about 50 mg/day in single or 2-4 divided doses.

In another alternative embodiment, the compositions or treatments of the present invention can comprise at least one (e.g., 1, 2 or 3, or 1 or 2, or 1, and usually 1) compound of Formula 1, or pharmaceutically acceptable salts or solvates thereof, and nicotinic acid (niacin) and/or derivatives thereof. Nicotinic acid and its derivatives inhibit hepatic production of VLDL and its metabolite LDL and increases HDL and apo A-1 levels. An example of a suitable nicotinic acid product is NIASPAN® (niacin extended-release tablets) which are available from Kos.

Generally, a total daily dosage of nicotinic acid or a derivative thereof can range from about 500 to about 10,000 mg/day, preferably about 1000 to about 8000 mg/day, and more preferably about 3000 to about 6000 mg/day in single or divided doses.

In another alternative embodiment, the compositions or treatments of the present invention can comprise at least one (e.g., 1, 2 or 3, or 1 or 2, or 1, and usually 1) compound of Formula 1, or pharmaceutically acceptable salts or solvates thereof, and one or more (e.g., 1, 2 or 3, or 1 or 2, or 1, and usually 1) AcylCoA: Cholesterol O-acyltransferase (“ACAT”) Inhibitors, which can reduce LDL and VLDL levels. ACAT is an enzyme responsible for esterifying excess intracellular cholesterol and may reduce the synthesis of VLDL, which is a product of cholesterol esterification, and overproduction of apo B-100-containing lipoproteins. Generally, a total daily dosage of ACAT inhibitor(s) can range from about 0.1 to about 1000 mg/day in single or 2-4 divided doses.

In another alternative embodiment, the compositions or treatments of the present invention can comprise at least one (e.g., 1, 2 or 3, or 1 or 2, or 1, and usually 1) compound of Formula 1, or pharmaceutically acceptable salts or solvates thereof, and one or more (e.g., 1, 2 or 3, or 1 or 2, or 1, and usually 1) Cholesteryl Ester Transfer Protein (“CETP”) Inhibitors. CETP is responsible for the exchange or transfer of cholesteryl ester carrying HDL and triglycerides in VLDL. Pancreatic cholesteryl ester hydrolase (pCEH) inhibitors such as WAY-121898 also can be co-administered with or in combination.

Generally, a total daily dosage of CETP inhibitor(s) can range from about 0.01 to about 1000 mg/day, and preferably about 0.5 to about 20 mg/kg body weight/day in single or divided doses.

In another alternative embodiment, the compositions or treatments of the present invention can comprise at least one (e.g., 1, 2 or 3, or 1 or 2, or 1, and usually 1) compound of Formula 1, or pharmaceutically acceptable salts or solvates thereof and probucol or derivatives thereof, which can reduce LDL levels.

Generally, a total daily dosage of probucol or derivatives thereof can range from about 10 to about 2000 mg/day, and preferably about 500 to about 1500 mg/day in single or 2-4 divided doses.

In another alternative embodiment, the compositions or treatments of the present invention can comprise at least one (e.g., 1, 2 or 3, or 1 or 2, or 1, and usually 1) compound of Formula 1, or pharmaceutically acceptable salts or solvates thereof, and low-density lipoprotein (LDL) receptor activators.

Generally, a total daily dosage of LDL receptor activator(s) can range from about 1 to about 1000 mg/day in single or 2-4 divided doses.

In another alternative embodiment, the compositions or treatments of the present invention can comprise at least one (e.g., 1, 2 or 3, or 1 or 2, or 1, and usually 1) compound of Formula 1, or pharmaceutically acceptable salts or solvates thereof, and fish oil. Generally, a total daily dosage of fish oil or Omega 3 fatty acids can range from about 1 to about 30 grams per day in single or 2-4 divided doses.

In another alternative embodiment, the compositions or treatments of the present invention can further comprise at least one (e.g., 1, 2 or 3, or 1 or 2, or 1, and usually 1) compound of Formula 1, or pharmaceutically acceptable salts or solvates thereof, and natural water soluble fibers, such as psyllium, guar, oat and pectin, which can reduce cholesterol levels. Generally, a total daily dosage of natural water soluble fibers can range from about 0.1 to about 10 grams per day in single or 2-4 divided doses.

In another alternative embodiment, the compositions or treatments of the present invention can comprise at least one (e.g., 1, 2 or 3, or 1 or 2, or 1, and usually 1) compound of Formula 1, or pharmaceutically acceptable salts or solvates thereof, and plant sterols, plant stanols and/or fatty acid esters of plant stanols, such as sitostanol ester used in BENECOL® margarine, which can reduce cholesterol levels. Generally, a total daily dosage of plant sterols, plant stanols and/or fatty acid esters of plant stanols can range from about 0.5 to about 20 grams per day in single or 2-4 divided doses.

In another alternative embodiment, the compositions or treatments of the present invention can comprise at least one (e.g., 1, 2 or 3, or 1 or 2, or 1, and usually 1) compound of Formula 1, or pharmaceutically acceptable salts, solvates, or esters thereof, and antioxidants, such as probucol, tocopherol, ascorbic acid, β-carotene and selenium, or vitamins such as vitamin B₆ or vitamin B₁₂. Generally, a total daily dosage of antioxidants or vitamins can range from about 0.05 to about 10 grams per day in single or 2-4 divided doses.

In another alternative embodiment, the compositions or treatments of the present invention can comprise at least one (e.g., 1, 2 or 3, or 1 or 2, or 1, and usually 1) compound of Formula 1, or pharmaceutically acceptable salts or solvates thereof, and monocyte and macrophage inhibitors such as polyunsaturated fatty acids (PUFA), thyroid hormones including throxine analogues such as CGS-26214 (a thyroxine compound with a fluorinated ring), gene therapy and use of recombinant proteins such as recombinant apo E, Generally, a total daily dosage of these agents can range from about 0.01 to about 1000 mg/day in single or 2-4 divided doses.

Also useful with the present invention are compositions or therapeutic combinations that further comprise hormone replacement agents and compositions. Useful hormone agents and compositions for hormone replacement therapy of the present invention include androgens, estrogens, progestins, their pharmaceutically acceptable salts and derivatives thereof. Combinations of these agents and compositions are also useful.

The dosage of androgen and estrogen combinations vary, desirably from about 1 mg to about 4 mg androgen and from about 1 mg to about 3 mg estrogen. Examples include, but are not limited to, androgen and estrogen combinations such as the combination of esterified estrogens (sodium estrone sulfate and sodium equilin sulfate) and methyltestosterone (17-hydroxy-17-methyl-, (17B)-androst-4-en-3-one) available from Solvay Pharmaceuticals, Inc., Marietta, Ga., under the tradename Estratest.

Estrogens and estrogen combinations may vary in dosage from about 0.01 mg up to 8 mg, desirably from about 0.3 mg to about 3.0 mg. Examples of useful estrogens and estrogen combinations include:

(a) the blend of nine (9) synthetic estrogenic substances including sodium estrone sulfate, sodium equilin sulfate, sodium 17α-dihydroequilin sulfate, sodium 17α-estradiol sulfate, sodium 17β-dihydroequilin sulfate, sodium 17α-dihydroequilenin sulfate, sodium 17β-dihydroequilenin sulfate, sodium equilenin sulfate and sodium 17β-estradiol sulfate; available from Duramed Pharmaceuticals, Inc., Cincinnati, Ohio, under the tradename Cenestin;

(b) ethinyl estradiol (19-nor-17α-pregna-1,3,5(10)-trien-20-yne-3,17-diol; available by Schering Plough Corporation, Kenilworth, N.J., under the tradename Estinyl;

(c) esterified estrogen combinations such as sodium estrone sulfate and sodium equilin sulfate; available from Solvay under the tradename Estratab and from Monarch Pharmaceuticals, Bristol, Tenn., under the tradename Menest;

(d) estrapipate (piperazine estra-1,3,5(10)-trien-17-one, 3-(sulfooxy)-estrone sulfate), available from Pharmacia & Upjohn, Peapack, N.J., under the tradename Ogen and from Women First Health Care, Inc., San Diego, Calif., under the tradename Ortho-Est; and

(e) conjugated estrogens (17α-dihydroequilin, 17α-estradiol, and 17β-dihydroequilin); available from Wyeth-Ayerst Pharmaceuticals, Philadelphia, Pa., under the tradename Premarin.

Progestins and estrogens may also be administered with a variety of dosages, generally from about 0.05 to about 2.0 mg progestin and about 0.001 mg to about 2 mg estrogen, desirably from about 0.1 mg to about 1 mg progestin and about 0.01 mg to about 0.5 mg estrogen. Examples of progestin and estrogen combinations that may vary in dosage and regimen include:

(a) the combination of estradiol (estra-1,3,5(10)-triene-3,17β-diol hemihydrate) and norethindrone (17β-acetoxy-19-nor-17α-pregn-4-en-20-yn-3-one); which is available from Pharmacia & Upjohn, Peapack, N.J., under the tradename Activella;

(b) the combination of levonorgestrel (d(−)-13β-ethyl-17α-ethinyl-17β-hydroxygon-4-en-3-one) and ethinyl estradial; available from Wyeth-Ayerst under the tradename Alesse, from Watson Laboratories, Inc., Corona, Calif., under the tradenames Levora and Trivora, Monarch Pharmaceuticals, under the tradename Nordeffe, and from Wyeth-Ayerst under the tradename Triphasil;

(c) the combination of ethynodiol diacetate (19-nor-17α-pregn-4-en-20-yne-3β,17-diol diacetate) and ethinyl estradiol; available from G.D. Searle & Co., Chicago, Ill., under the tradename Demulen and from Watson under the tradename Zovia;

(d) the combination of desogestrel (13-ethyl-11-methylene-18,19-dinor-17α-pregn-4-en-20-yn-17-ol) and ethinyl estradiol; available from Organon under the tradenames Desogen and Mircette, and from Ortho-McNeil Pharmaceutical, Raritan, N.J., under the tradename Ortho-Cept;

(e) the combination of norethindrone and ethinyl estradiol; available from Parke-Davis, Morris Plains, N.J., under the tradenames Estrostep and FemHRT, from Watson under the tradenames Microgestin, Necon, and Tri-Norinyl, from Ortho-McNeil under the tradenames Modicon and Ortho-Novum, and from Warner Chilcoft Laboratories, Rockaway, N.J., under the tradename Ovcon;

(f) the combination of norgestrel ((±)-13-ethyl-17-hydroxy-18,19-dinor-17α-preg-4-en-20-yn-3-one) and ethinyl estradiol; available from Wyeth-Ayerst under the tradenames Ovral and Lo/Ovral, and from Watson under the tradenames Ogestrel and Low-Ogestrel;

(g) the combination of norethindrone, ethinyl estradiol, and mestranol (3-methoxy-19-nor-17α-pregna-1,3,5(10)-trien-20-yn-17-ol); available from Watson under the tradenames Brevicon and Norinyl;

(h) the combination of 17β-estradiol(estra-1,3,5(10)-triene-3,17β-diol) and micronized norgestimate (17α-17-(Acetyloxyl)-3-ethyl-18,19-dinorpregn-4-en-20-yn-3-one-3-oxime); available from Ortho-McNeil under the tradename Ortho-Prefest;

(i) the combination of norgestimate (18,19-dinor-17-pregn-4-en-20-yn-3-one, 17--(acetyloxy)-13-ethyl-,oxime, (17(α)-(+)-) and ethinyl estradiol; available from Ortho-McNeil under the tradenames Ortho Cyclen and Ortho Tri-Cyclen; and

(j) the combination of conjugated estrogens (sodium estrone sulfate and sodium equilin sulfate) and medroxyprogesterone acetate (20-dione, 17-(acetyloxy)-6-methyl-, (6(α))- pregn-4-ene-3); available from Wyeth-Ayerst under the tradenames Premphase and Prempro.

In general, a dosage of progestins may vary from about 0.05 mg to about 10 mg or up to about 200 mg if microsized progesterone is administered. Examples of progestins include norethindrone; available from ESI Lederle, Inc., Philadelphia, Pa., under the tradename Aygestin, from Ortho-McNeil under the tradename Micronor, and from Watson under the tradename Nor-QD; norgestrel; available from Wyeth-Ayerst under the tradename Ovrette; micronized progesterone (pregn-4-ene-3,20-dione); available from Solvay under the tradename Prometrium; and medroxyprogesterone acetate; available from Pharmacia & Upjohn under the tradename Provera.

In another alternative embodiment, the compositions, therapeutic combinations or methods of the present invention can comprise at least one (e.g., 1, 2 or 3, or 1 or 2, or 1, and usually 1) compound of Formula 1, or pharmaceutically acceptable salts or solvates thereof, and one or more (e.g., 1, 2 or 3, or 1 or 2, or 1, and usually 1) obesity control medications. Useful obesity control medications include, but are not limited to, drugs that reduce energy intake or suppress appetite, drugs that increase energy expenditure and nutrient-partitioning agents. Suitable obesity control medications include, but are not limited to, noradrenergic agents (such as diethylpropion, mazindol, phenylpropanolamine, phentermine, phendimetrazine, phendamine tartrate, methamphetamine, phendimetrazine and tartrate); serotonergic agents (such as sibutramine, fenfluramine, dexfenfluramine, fluoxetine, fluvoxamine and paroxtine); thermogenic agents (such as ephedrine, caffeine, theophylline, and selective β3-adrenergic agonists); alpha-blocking agents; kainite or AMPA receptor antagonists; leptin-lipolysis stimulated receptors; phosphodiesterase enzyme inhibitors; compounds having nucleotide sequences of the mahogany gene; fibroblast growth factor-10 polypeptides; monoamine oxidase inhibitors (such as befloxatone, moclobemide, brofaromine, phenoxathine, esuprone, befol, toloxatone, pirlindol, amiflamine, sercloremine, bazinaprine, lazabemide, milacemide and caroxazone); compounds for increasing lipid metabolism (such as evodiamine compounds); and lipase inhibitors (such as orlistat). Generally, a total dosage of the above-described obesity control medications can range from 1 to 3,000 mg/day, desirably from about 1 to 1,000 mg/day and more desirably from about 1 to 200 mg/day in single or 2-4 divided doses.

The compositions, therapeutic combinations or methods of the present invention can comprise at least one (e.g., 1, 2 or 3, or 1 or 2, or 1, and usually 1) compound of Formula 1, or pharmaceutically acceptable salts or solvates thereof, and one or more (e.g., 1, 2 or 3, or 1 or 2, or 1, and usually 1) blood modifiers which are chemically different from the substituted azetidinone and substituted β-lactam compounds and the lipid modulating agents discussed above, for example, they contain one or more different atoms, have a different arrangement of atoms or a different number of one or more atoms than the sterol absorption inhibitor(s) or lipid modulating agents discussed above. Useful blood modifiers include but are not limited to anti-coagulants (argatroban, bivalirudin, dalteparin sodium, desirudin, dicumarol, lyapolate sodium, nafamostat mesylate, phenprocoumon, tinzaparin sodium, warfarin sodium); antithrombotic (anagrelide hydrochloride, bivalirudin, cilostazol, dalteparin sodium, danaparoid sodium, dazoxiben hydrochloride, efegatran sulfate, enoxaparin sodium, fluretofen, ifetroban, ifetroban sodium, lamifiban, lotrafiban hydrochloride, napsagatran, orbofiban acetate, roxifiban acetate, sibrafiban, tinzaparin sodium, trifenagrel, abciximab, zolimomab aritox); fibrinogen receptor antagonists (roxifiban acetate, fradafiban, orbofiban, lotrafiban hydrochloride, tirofiban, xemilofiban, monoclonal antibody 7E3, sibrafiban); platelet inhibitors (cilostazol, clopidogrel bisulfate, epoprostenol, epoprostenol sodium, ticlopidine hydrochloride, aspirin, ibuprofen, naproxen, sulindae, idomethacin, mefenamate, droxicam, diclofenac, sulfinpyrazone, piroxicam, dipyridamole); platelet aggregation inhibitors (acadesine, beraprost, beraprost sodium, ciprostene calcium, itazigrel, lifarizine, lotrafiban hydrochloride, orbofiban acetate, oxagrelate, fradafiban, orbofiban, tirofiban, xemilofiban); hemorrheologic agents (pentoxifylline); lipoprotein associated coagulation inhibitors; Factor VIIa inhibitors (4H-3,1-benzoxazin-4-ones, 4H-3,1-benzoxazin-4-thiones, quinazolin-4-ones, quinazolin-4-thiones, benzothiazin-4-ones, imidazolyl-boronic acid-derived peptide analogues TFPI-derived peptides, naphthalene-2-sulfonic acid {1-[3-(aminoiminomethyl)-benzyl]-2-oxo-pyrrolidin-3-(S)-yl}amide trifluoroacetate, dibenzofuran-2-sulfonic acid {1-[3-(aminomethyl)-benzyl]-5-oxo-pyrrolidin-3-yl}-amide, tolulene-4-sulfonic acid {1-[3-(aminoiminomethyl)-benzyl]-2-oxo-pyrrolidin-3-(S)-yl}-amide trifluoroacetate, 3,4-dihydro-1H-isoquinoline-2-sulfonic acid {1-[3-(aminoiminomethyl)-benzyl]-2-oxo-pyrrolin-3-(S)-yl}-amide trifluoroacetate); Factor Xa inhibitors (disubstituted pyrazolines, disubstituted triazolines, substituted n-[(aminoiminomethyl)phenyl]propylamides, substituted n-[(aminomethyl)phenyl]propylamides, tissue factor pathway inhibitor (TFPI), low molecular weight heparins, heparinoids, benzimidazolines, benzoxazolinones, benzopiperazinones, indanones, dibasic (amidinoaryl) propanoic acid derivatives, amidinophenyl-pyrrolidines, amidinophenyl-pyrrolines, amidinophenyl-isoxazolidines, amidinoindoles, amidinoazoles, bis-arlysulfonylaminobenzamide derivatives, peptidic Factor Xa inhibitors).

The compositions, therapeutic combinations or methods of the present invention can comprise at least one (e.g., 1, 2 or 3, or 1 or 2, or 1, and usually 1) compound of Formula 1, or pharmaceutically acceptable salts or solvates thereof, and one or more (e.g., 1, 2 or 3, or 1 or 2, or 1, and usually 1) cardiovascular agents which are chemically different from the substituted azetidinone and substituted β-lactam compounds and the lipid modulating agents discussed above, for example, they contain one or more different atoms, have a different arrangement of atoms or a different number of one or more atoms than the sterol absorption inhibitor(s) or PPAR receptor activators discussed above. Useful cardiovascular agents include but are not limited to calcium channel blockers (clentiazem mateate, amlodipine besylate, isradipine, nimodipine, felodipine, nilvadipine, nifedipine, teludipine hydrochloride, diltiazem hydrochloride, belfosdil, verapamil hydrochloride, fostedil); adrenergic blockers (fenspiride hydrochloride, labetalol hydrochloride, proroxan, alfuzosin hydrochloride, acebutolol, acebutolol hydrochloride, alprenolol hydrochloride, atenolol, bunolol hydrochloride, carteolol hydrochloride, celiprolol hydrochloride, cetamolol hydrochloride, cicloprolol hydrochloride, dexpropranolol hydrochloride, diacetolol hydrochloride, dilevalol hydrochloride, esmolol hydrochloride, exaprolol hydrochloride, flestolol sulfate, labetalol hydrochloride, levobetaxolol hydrochloride, levobunolol hydrochloride, metalol hydrochloride, metoprololt, metoprolol tartrate, nadolol, pamatolol sulfate, penbutolol sulfate, practolol, propranolol hydrochloride, sotalol hydrochloride, timolol, timolol maleate, tiprenolol hydrochloride, tolamolol, bisoprolol, bisoprolol fumarate, nebivolol); adrenergic stimulants; angiotensin converting enzyme (ACE) inhibitors (benazepril hydrochloride, benazeprilat, captopril, delapril hydrochloride, fosinopril sodium, libenzapril, moexipril hydrochloride, pentopril, perindopril, quinapril hydrochloride, quinaprilat, ramipril, spirapril hydrochloride, spiraprilat, teprotide, enalapril maleate, lisinopril, zofenopril calcium, perindopril erbumine); antihypertensive agents (althiazide, benzthiazide, captopril, carvedilol, chlorothiazide sodium, clonidine hydrochloride, cyclothiazide, delapril hydrochloride, dilevalol hydrochloride, doxazosin mesylate, fosinopril sodium, guanfacine hydrochloride, methyldopa, metoprolol succinate, moexipril hydrochloride, monatepil maleate, pelanserin hydrochloride, phenoxybenzamine hydrochloride, prazosin hydrochloride, primidolol, quinapril hydrochloride, quinaprilat, ramipril, terazosin hydrochloride, candesartan, candesartan cilexetil, telmisartan, amlodipine besylate, amlodipine maleate, bevantolol hydrochloride); angiotensin II receptor antagonists (candesartan, irbesartan, losartan potassium, candesartan cilexetit, telmisartan); anti-anginal agents (amlodipine besylate, amlodipine maleate, betaxolol hydrochloride, bevantolol hydrochloride, butoprozine hydrochloride, carvedilol, cinepazet maleate, metoprolol succinate, molsidomine, monatepil maleate, primidolol, ranolazine hydrochloride, tosifen, verapamil hydrochloride); coronary vasodilators (fostedil, azaclorzine hydrochloride, chromonar hydrochloride, clonitrate, diltiazem hydrochloride, dipyridamole, droprenilamine, erythrityl tetranitrate, isosorbide dinitrate, isosorbide mononitrate, lidoflazine, mioflazine hydrochloride, mixidine, molsidomine, nicorandil, nifedipine, nisoldipine, nitroglycerine, oxprenolol hydrochloride, pentrinitrol, perhexiline maleate, prenylamine, propatyl nitrate, terodiline hydrochloride, tolamolol, verapamil); diuretics (the combination product of hydrochlorothiazide and spironolactone and the combination product of hydrochlorothiazide and triamterene).

The compositions, therapeutic combinations or methods of the present invention can comprise at least one (e.g., 1, 2 or 3, or 1 or 2, or 1, and usually 1) compound of Formula 1, or pharmaceutically acceptable salts or solvates thereof, and one or more (e.g., 1, 2 or 3, or 1 or 2, or 1, and usually 1) antidiabetic medications for reducing blood glucose levels in a human. Useful antidiabetic medications include, but are not limited to, drugs that reduce energy intake or suppress appetite, drugs that increase energy expenditure and nutrient-partitioning agents. Suitable antidiabetic medications include, but are not limited to, sulfonylurea (such as acetohexamide, chlorpropamide, gliamilide, gliciazide, glimepiride, glipizide, glyburide, glibenclamide, tolazamide, and tolbutamide), meglitinide (such as repaglinide and nateglinide), biguanide (such as metformin and buformin), alpha-glucosidase inhibitor (such as acarbose, miglitol, carniglibose, and voglibose), certain peptides (such as amlintide, pramlintide, exendin, and GLP-1 agonistic peptides), and orally administrable insulin or insulin composition for intestinal delivery thereof. Generally, a total dosage of the above-described antidiabetic medications can range from 0.1 to 1,000 mg/day in single or 2-4 divided doses.

Mixtures of two, three, four or more of any of the pharmacological or therapeutic agents described above can be used in the compositions and therapeutic combinations of the present invention.

In yet another embodiment, the present invention provides a method of treating, reducing, or ameliorating a disease or condition selected from the group consisting of metabolic syndrome (e.g., abdominal obesity, atherogenic dyslipidemia, insulin resistance and glucose intolerance), insulin sensitivity, neuroinflammatory disorders, cognitive disorders, psychosis, addictive behavior, gastrointestinal disorders, vascular conditions, hyperlipidaemia, atherosclerosis, hypercholesterolemia, sitosterolemia, vascular inflammation, stroke, diabetes, and cardiovascular conditions, and/or reduce the level of sterol(s) in a patient in need thereof, comprising administering to said patient an effective amount of at least one (e.g., 1, 2 or 3, or 1 or 2, or 1, and usually 1) compound of Formula 1, or a pharmaceutically acceptable salt or solvate thereof, and one or more (e.g., 1, 2 or 3, or 1 or 2, or 1) cholesterol lowering compound.

The treatment compositions and therapeutic combinations comprising at least one compound of Formula 1 and at least one cholesterol lowering agent can inhibit the intestinal absorption of cholesterol in mammals can be useful in the treatment and/or prevention of conditions, for example vascular conditions, such as atherosclerosis, hypercholesterolemia and sitosterolemia, stroke, obesity and lowering of plasma levels of cholesterol in mammals, in particular in mammals.

In another embodiment of the present invention, the compositions and therapeutic combinations of the present invention can inhibit sterol or 5α-stanol absorption or reduce plasma concentration of at least one sterol selected from the group consisting of phytosterols (such as sitosterol, campesterol, stigmasterol and avenosterol) and/or 5α-stanol (such as cholestanol, 5α-campestanol, 5α-sitostanol), cholesterol and mixtures thereof. The plasma concentration can be reduced by administering to a mammal in need of such treatment an effective amount of at least one treatment composition or therapeutic combination comprising at least one selective CB₁ receptor antagonist and at least one cholesterol lowering compound, for example a sterol absorption inhibitor described above. The reduction in plasma concentration of sterols or 5α-stanols can range from about 1 to about 70 percent, and preferably about 10 to about 50 percent. Methods of measuring serum total blood cholesterol and total LDL cholesterol are well known to those skilled in the art and for example include those disclosed in PCT WO 99/38498 at page 11, incorporated by reference herein. Methods of determining levels of other sterols in serum are disclosed in H. Gyiling et al., “Serum Sterols During Stanol Ester Feeding in a Mildly Hypercholesterolemic Population”, J. Lipid Res. 40: 593-600 (1999), incorporated by reference herein.

The treatments of the present invention can also reduce the size or presence of plaque deposits in vascular vessels. The plaque volume can be measured using (IVUS), in which a tiny ultrasound probe is inserted into an artery to directly image and measure the size of atherosclerotic plaques, in a manner well know to those skilled in the art.

Since the present invention relates to treating conditions as discussed above, by treatment with a combination of active ingredients wherein the active ingredients may be administered separately, the invention also relates to combining separate pharmaceutical compositions in kit form. That is, a kit is contemplated wherein two separate units are combined: a pharmaceutical composition comprising at least one selective CB₁ receptor antagonist of Formula 1, or a pharmaceutically acceptable salt or solvate thereof and a separate pharmaceutical composition comprising at least one cholesterol lowering compound as described above. The kit will preferably include directions for the administration of the separate components. The kit form is particularly advantageous when the separate components must be administered in different dosage forms (e.g., oral and parenteral) or are administered at different dosage intervals.

The compounds of the present invention, e.g., according to Formula 1, are preferably purified to a degree suitable for use as a pharmaceutically active substance. That is, the compounds of Formula 1 can have a purity of 95 wt % or more (excluding adjuvants such as pharmaceutically acceptable carriers, solvents, etc., which are used in formulating the compound of Formula 1 into a conventional form, such as a pill, capsule, IV solution, etc. suitable for administration into a patient). More preferably, the purity can be 97 wt % or more, even more preferably, 99 wt % or more. A purified compound of Formula 1 includes a single isomer having a purity, as discussed above, of 95 wt % or more, 97 wt % or more, or 99 wt % or more, as discussed above. For example, the purified compound of Formula 1 can have a purity of 95 wt % or more, 97 wt % or more, or 99 wt % or more.

Alternatively, the purified compound of Formula 1 can include a mixture of isomers, each having a structure according to Formula 1, where the amount of impurity (i.e., compounds or other contaminants, exclusive of adjuvants as discussed above) is 5 wt % or less, 3 wt % or less, or 1 wt % or less. For example, the purified compound of Formula 1 can be an isomeric mixture of compounds of Formula 1, where the ratio of the amounts of the two isomers is approximately 1:1, and the combined amount of the two isomers is 95 wt % or more, 97 wt % or more, or 99 wt % or more.

The invention disclosed herein is exemplified by the following preparations and examples which should not be construed to limit the scope of the disclosure. Alternative mechanistic pathways and analogous structures will be apparent to those skilled in the art. 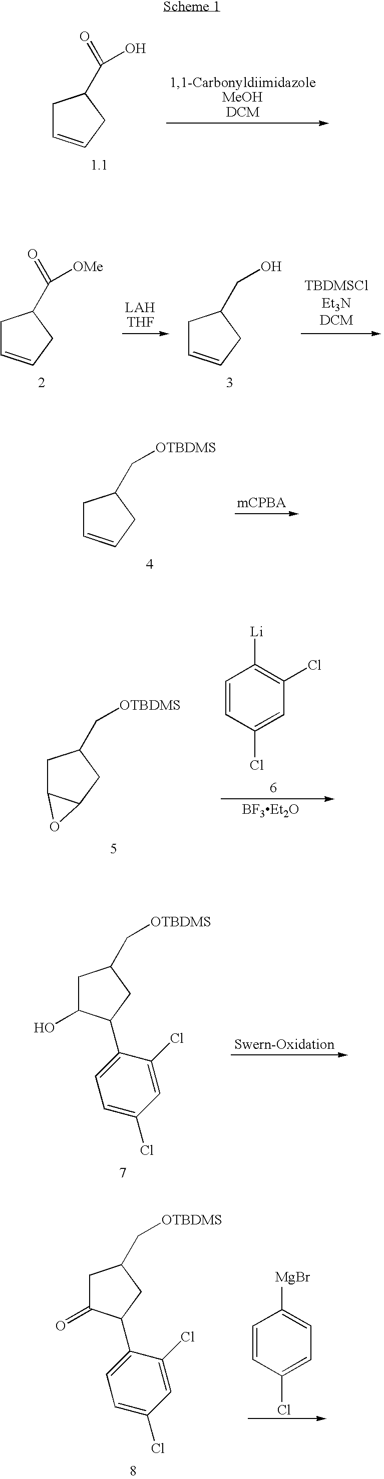 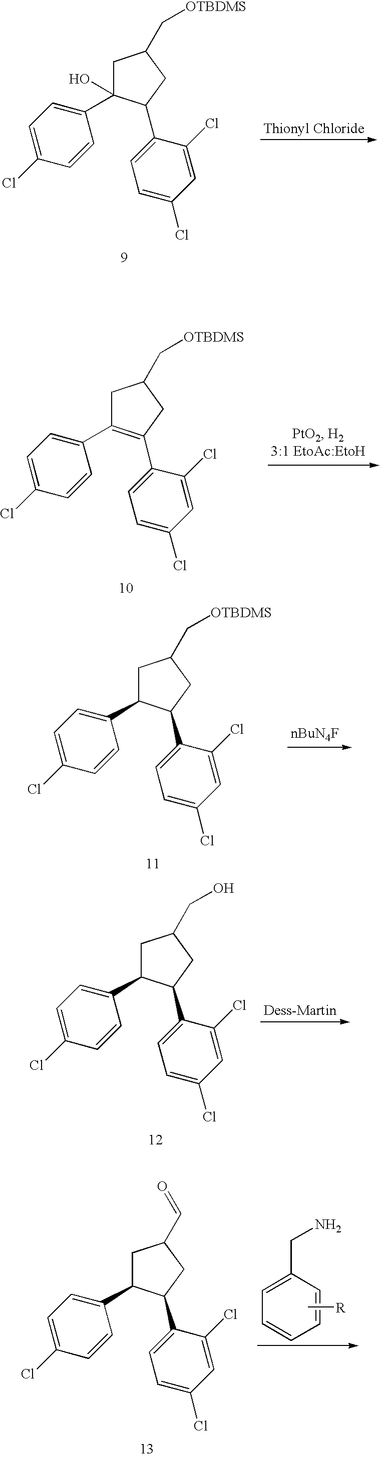 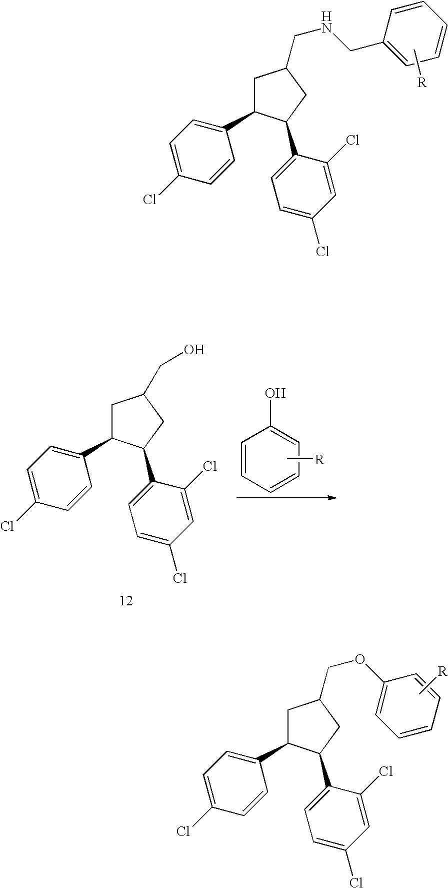 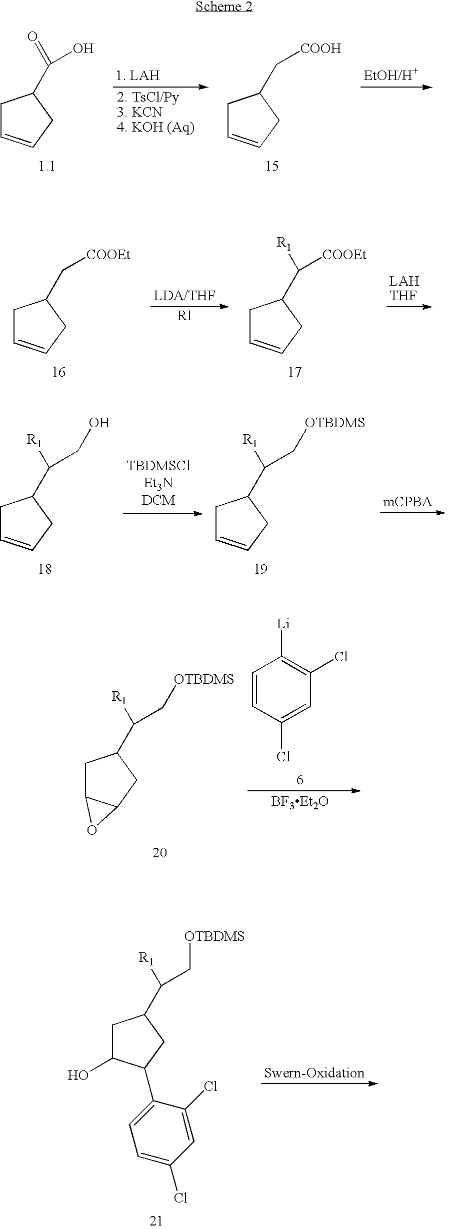 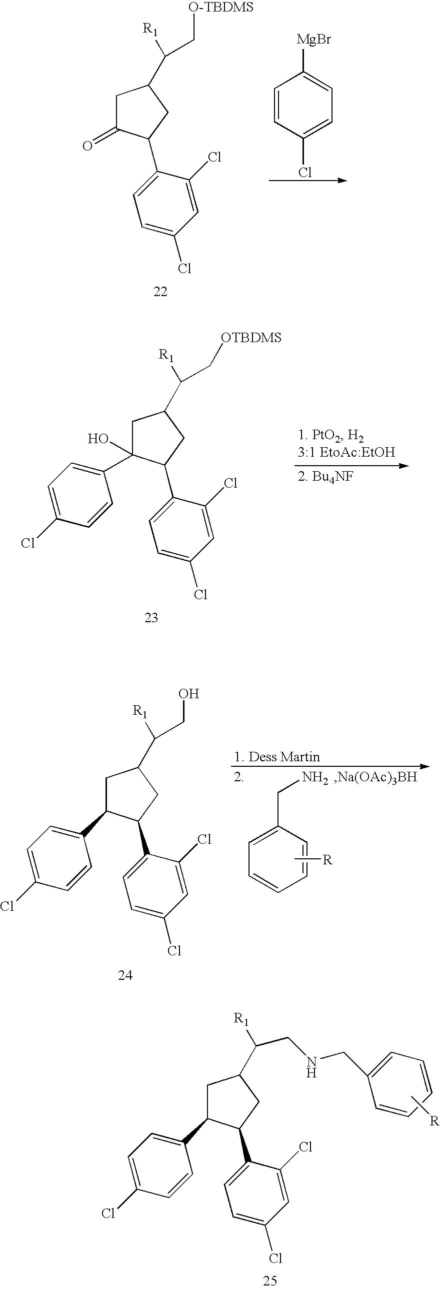 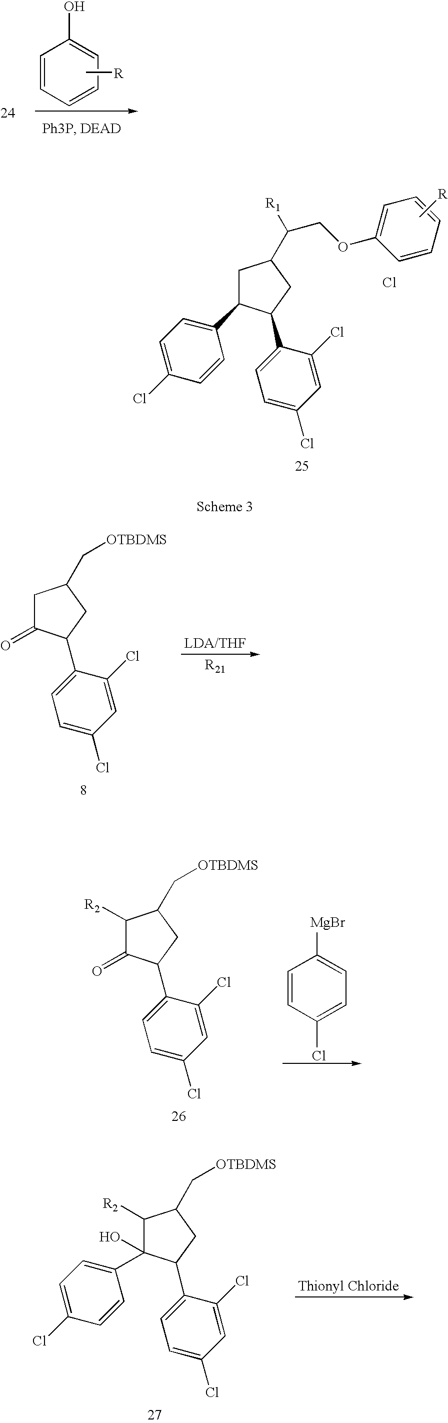 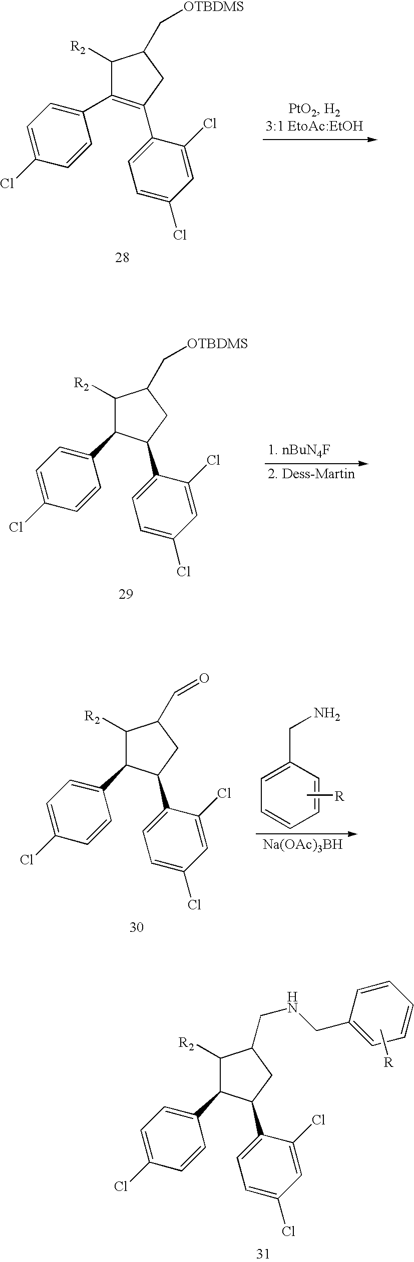 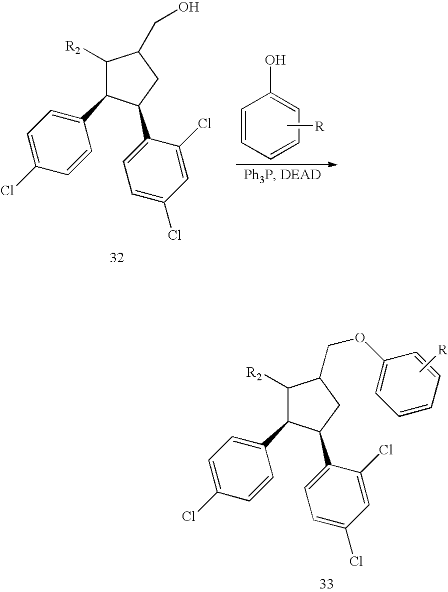
Procedure:

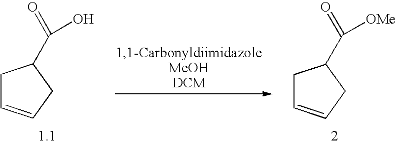

To a solution of 1.1 (10 g, 89 mmol) in DCM (100 mL) at 0° C. under N₂ atmosphere was added 1,1-carbonyldiimidazole (43 g, 267 mmol). The mixture was stirred at room temperature for 4 hours, then MeOH (100 mL) was added. The mixture was allowed to stir overnight. The solvent was evaporated at low temperature to afford 2 (11.2 g, 99%).

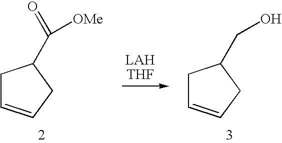

To a solution of 2 (8.6 g, 68 mmol) in THF (40.0 mL) at 0° C. was added LAH (54 mL, 1M solution in THF) under N₂ atmosphere. The mixture was stirred at ambient temperature for 18 hours. The reaction mixture was quenched with water and NaOH (10 w/t %). The mixture was filtered through celite and solvent was removed to give compound 3 (6.6 g, 100%).

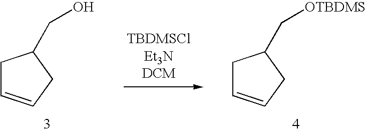

A mixture of 3 (4.4 g, 45 mmol), TBDMSCI (7.5 g, 50 mmol) and Et₃N (19 mL, 135 mmol) in DCM (45 mL) was stirred at room temperature under N₂ atmosphere for overnight. The mixture was washed with saturated aqueous NaHCO₃ solution and the organic layer was extracted with DCM. The combined extracts were dried over MgSO₄. The crude residue was purified via silica gel column chromatography (100% Hexane) to give 4 (7.4 g, 79%).

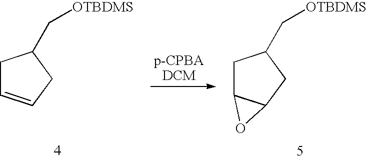

To a solution of 4 (3.7 g, 17.5 mmol) in DCM (50 mL) under N₂ atmosphere was added 3-Chloroperoxybenzoic acid (7.8 g, 26.0 mmol). The mixture was stirred at ambient temperature for 4 hours. The saturated aqueous solution of Na₂S₂O₃ was added to the mixture and stirred for 15 minutes. The organic layer was extracted with DCM. The combined extracts were washed with saturated aqueous solution of NaHCO₃ and dried over MgSO₄. The crude residue was purified via silica gel column chromatograph (Ethylacetate:Hexane=10:90) to give 5 (3.8 g, 96%).

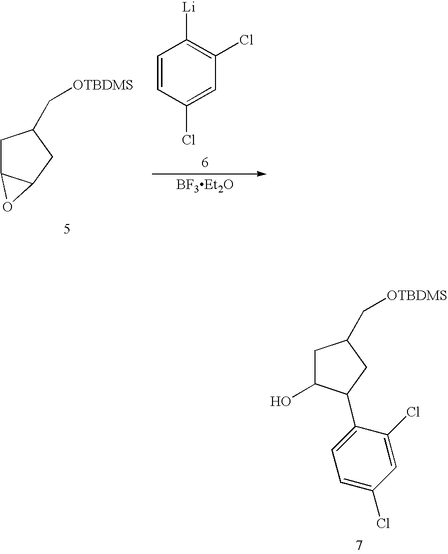

A solution of 6 was prepared by treating 1,3-dichloro-4-iodobenzene (3.5 g, 12.7 mmol) in EtO₂:THF (35.0 mL:17.5 mL, 2:1) at −78° C. under N₂ atmosphere with n-BuLi (5.6 mL, 14 mmol). To a solution of freshly prepared 6 was added 5 (2.9 g, 12.7 mmol) followed by BF₃.Et₂O (2.4 mL, 20 mmol). After stirring for 3 hours at −78° C., the temperature was brought to −40° C. and quenched the mixture with an ice-cold saturated aqueous NH₄Cl solution. The organic layer was extracted with EtO₂ and dried over MgSO₄. The solvent was removed at reduced pressure and the residue was purified via silica gel column chromatograph (Ethylacetate:Hexane=5:95) to afford 7 (3.1 g, 69%).

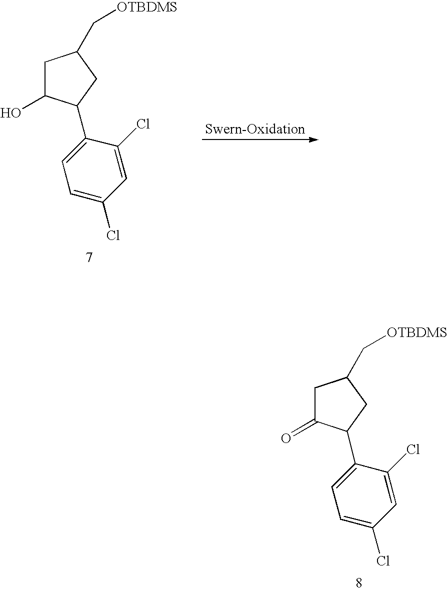

DMSO (1. 8mL, 25 mmol) was added to a flask containing Oxalyl chloride (1.2 mL, 14 mmol) in DCM (10 mL) at −78° C. under Argon atmosphere. The resulting mixture was stirred for 30 minutes, then 7 (2.6 g, 7.0 mmol) in DCM (7 mL) was added dropwise. After 2 hours of stirring, Et₃N (1.3 mL, 9.4 mmol) was added and stirred for another hour at ambient temperature. The reaction mixture was poured into H₂O and extracted with DCM. The combined extracts were dried over MgSO₄ and purified by column chromatography (Ethylacetate:Hexane=5:95) to yield 8 (2.3 g, 74%).

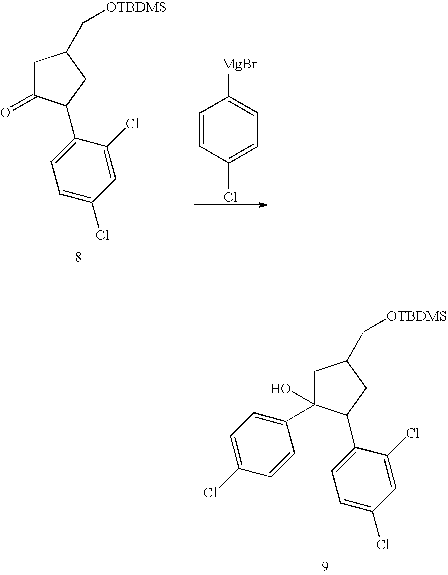

To a solution of 8 (98 mg, 0.26 mmol) in EtO₂ (1.5 mL) at 0° C. under N₂ atmosphere was added 4-Chlorophenyl magnesium bromide (0.53 mL, 1M solution in Et₂O). The resulting mixture was stirred for 30 minutes at 0° C., then it was allowed to warm up to room temperature and stirred for 6½ h. The mixture was washed with brine, extracted with Et₂O, dried over MgSO₄ and concentrated. The residue was purified via Prep plate TLC (Ethylacetate:Hexane=3:97) to give 9 (55 mg, 44%).

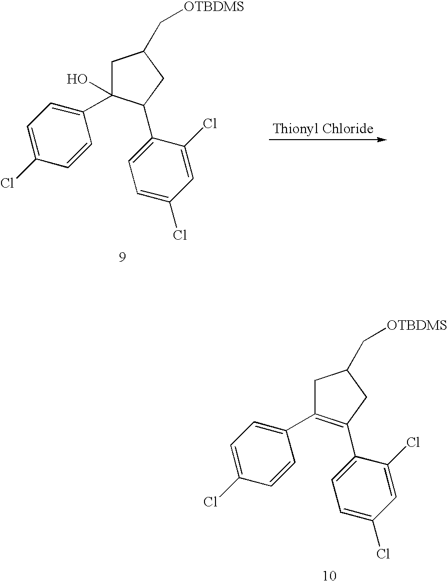

A solution of 9 (55 mg, 0.12 mmol) and Et₃N (0.09 mL, 0.18 mmol) was treated with thionyl chloride (0.01mL, 0.18 mmol) at room temperature under N₂ atmosphere, The resulting mixture was allowed to stir overnight. The mixture was diluted with DCM and washed with saturated aqueous NaHCO₃ solution. The combined extracts were dried over MgSO₄ and concentrated. The residue was purified via Prep Plate TLC (Ethylacetate:Hexane=2:98) to give 10 (39 mg, 69%).

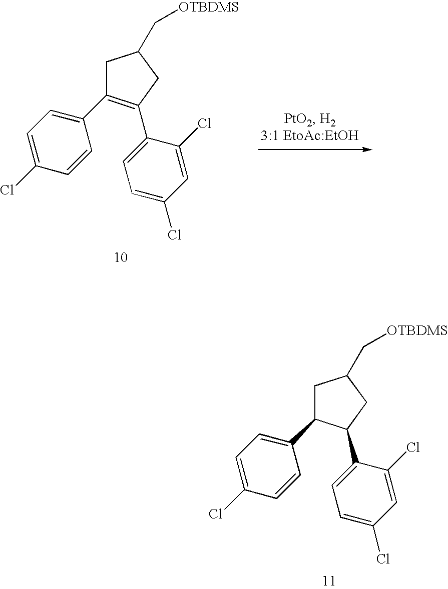

The compound 10 (50 mg, 0.11 mmol) was reduced using PtO₂ (14 mg, 0.06 mmol) in EtOAc/EtOH (3 mL:1 mL) under H₂ to give 11 (22 mg, 43%).

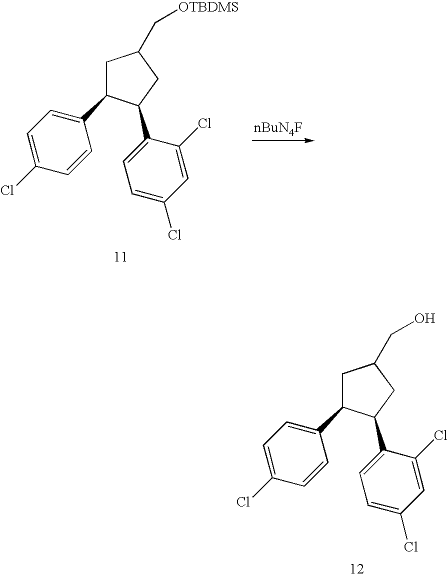

To a solution of 11 (22 mg, 0.05 mmol) in THF (2.0 mL) under N₂ atmosphere was added nBuN₄F (0.1 mL, 1M in THF solution). The mixture was stirred at room temperature overnight. The concentrated residue was purified via Prep Plate TLC (Ethylacetate:Hexane=25:75) to afford 12 (15 mg, 85%).

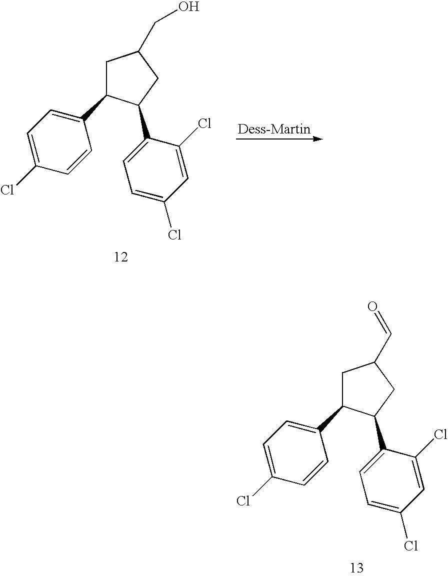

To a solution of 12 (50 mg, 0.14 mmol) in DCM at 0° C. under N₂ atmosphere was added Dess-martin periodinane (89 mg, 0.21 mmol). After 2 hours of stirring at ambient temperature, saturated aqueous Na₂S₂O₃ solution was added. After 10 minutes stirring, saturated aqueous NaHCO₃ solution was added. After 45 minutes of stirring, the organic layer was extracted with DCM and dried over MgSO₄. The residue was purified on the Prep Plate TLC using Ethyl acetate/Hexane (17:83) to give 13 (49 mg, 100%).

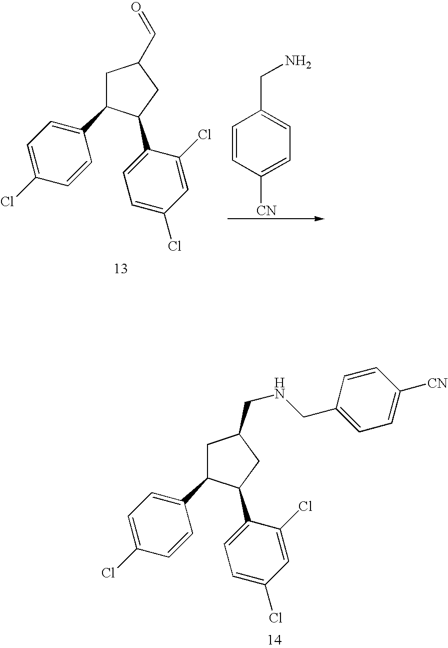

To a solution of 13 (26 mg, 0.07 mmol) in 1,2-Dichloroethane (2.0 mL) was added 4-Aminomethyl-benzonitrile (20 mg, 0.15 mmol). The mixture was stirred for 30 minutes under N₂ atmosphere, then NaBH(OAc)₃ (20 mg, 0.15 mmol) was added. After 20 hours of stirring, the organic mixture was washed with H₂O and extracted with DCM. The combined extracts were dried over MgSO₄ and concentrated. The residue was purified via Prep Plate TLC (MeOH:DCM=2:98) to give 14 (2.3 mg, 7%, MS m/e 469.1(M+1)). Compound 14 had a MS of 469 (M+1).

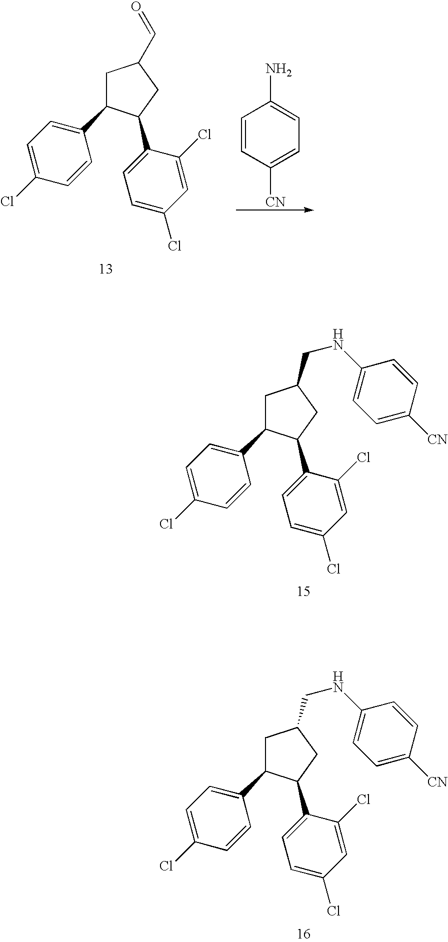

To a solution of 13 (30 mg, 0.09 mmol) in 1,2-Dichloroethane (2.0 mL) was added 4-aminobenzonitrile (21 mg, 0.18 mmol). The reaction mixture was stirred at room temperature under N₂ atmosphere over night. The solvent was removed under vacuo and the residue was purified on the Prep Plate TLC (Ethyl acetae:Hexane=20:80) to afford 15 (11.2 mg, 30%, MS m/e 455.1 (M+1)) and 16 (9 mg, 25%, MS m/e 455.1 (M+1)). Compound 15 had a MS of 455 (M+1). Compound 16 had a MS of 455 (M+1).

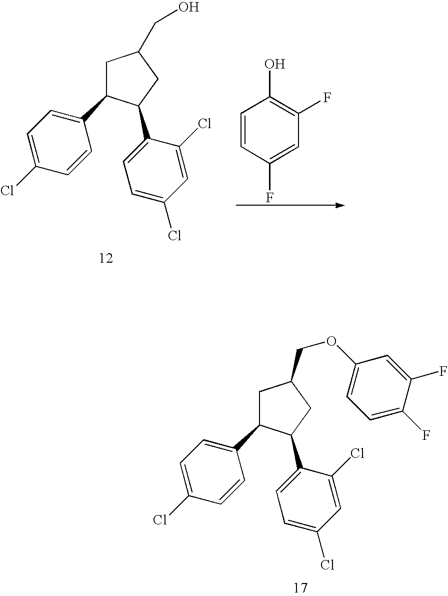

To a stirring solution of 12 (25 mg, 0.07 mmol), 3,4-difluorophenol (10.0 mg, 0.08 mmol) and PPh₃ (37 mg, 0.14 mmol) in THF (2.0 mL) at 0° C. under N₂ atmosphere was added DEAD (0.03 mL, 0.17 mmol) dropwise. The reaction mixture was stirred overnight and the solvent was removed under vacuo. The residue was purified on the Prep Plate TLC (Ethyl acetate:Hexane=17:83) to give 17 (19 mg, 57%, MS m/e 397.2 (M−2Cl)) Compound 17 had a MS of 397 (M−2Cl).

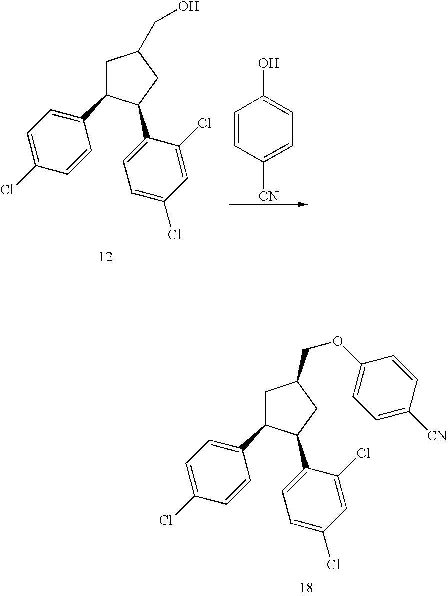

To a stirring solution of 12 (25 mg, 0.07 mmol), 4-cyanophenol (11 mg, 0.09 mmol) and PPh₃ (42 mg, 0.16 mmol) in THF (20 mL) at 0° C. under N₂ atmosphere was added DEAD (0.03 mL, 0.19 mmol) dropwise. The reaction mixture was stirred for 2½ hours at room temperature and the solvent was removed under vacuo. The residue was purified on the Prep Plate TLC (Ethyl acetate:Hexane=10:90) to afford 18 (18 mg, 50%, MS m/e 456.3 (M+1)) Compound 18 had a MS of 456 (M+1).

Assay

Competition binding assays for cannabinoid CB₁ and CB₂ affinity were performed by incubating commercially purchased membranes prepared from cells expressing each receptor subtype (8 μg pro) with 0.5 nM ³H-CP55,940, a non-selective cannabinoid agonist, along with concentrations of drug ranging from 0.0001-3 μM in Buffer A (5 mM MgCl₂, 2.5 mM EDTA and 013% BSA). Non-specific binding was defined in the presence of 10 μM CP55,940. For saturation studies, concentrations of ³H-CP55,940 ranging from 0.1-5 nM were incubated with membranes in the presence and absence of 10 μM CP55,940. Assays were terminated after incubation for 1½ hours by rapid filtration onto 0.3% polyethylenamine treated GF/C filterplates using a BRANDEL cell harvester. The plates were dried and MICROSCINT scintillation cocktail was added, after which the bound radioactivity was quantified using a TOPCOUNT scintillation counter.

The dissociation constant (K_{d}) of ³H-CP55,940 at the CB₁ and CB₂ receptor were determined by plotting specific binding at each concentration of radioligand, and analysis by non-linear regression. For competition studies, the concentration of each drug that inhibited 50 percent of ³H-CP55,940 binding (IC₅₀) was determined by non-linear regression analysis of the radioligand displacement curves. Affinity constants (Kᵢ) were calculated using the equation derived by Cheng and Prusoff (1973), defined as: IC₅₀/1+[conc. ligand/K_{d}].

### GTPγS Binding Protocol

The functional efficacy of compounds to activate second messengers within the cell was determined utilizing the GTPγS binding assay. Guanine nucleotides are phosphorylated within the plasma membrane of the cell following binding and activation by agonists. A radiolabelled derivative of guanine triphosphate (GTP) is utilized in this assay as it cannot be dephosphorylated and therefore accumulates following agonist binding. The simultaneous presence of an antagonist into this system will shift the agonist concentration curve to the right, with increasing concentrations of antagonist producing a greater rightward shift in the dose-response curve of the agonist.

Commercially purchased membranes were incubated with 10 mM GDP to allow sufficient substrate for phosphorylation in the presence of agonist. The membranes were then pre-incubated with increasing concentrations of test compound for 30 minutes to determine if they were capable of stimulating phosphorylation alone. Increasing concentrations of the non-selective cannabinoid agonist WIN55,122 were then added in the presence or absence of each concentration of test compound. The assay was then incubated for 1 hour at room temperature. To complete the assay, ³⁵S-GTPγS was added and the assay incubated for another 30 minutes. Assays were terminated by rapid filtration onto 10 mM sodium phosphate-treated GF/C filterplates using a BRANDEL cell harvester. The plates were dried and Microscint scintillation cocktail was added, after which the bound radioactivity was quantified using a TOPCOUNT scintillation counter.

The stimulation of ³⁵S-GTPγS binding as a function of the concentration of the agonist WIN55,122, in the absence and presence of test compound, was plotted and the EC₅₀ determined by nonlinear regression analysis using GraphPad Prism software. A Schild analysis of the rightward shift in the dose response curve of WIN55,122 in the presence of test compound was determined by plotting the concentration of test compound against the negative log of the dose ratio [1−(EC₅₀ agonist+test compound/EC50 of agonist alone)]. A linear regression analysis yields the Kb, defined as the X-intercept of the linear equation.

The CB1 Ki for Compounds 14, 15, 18, 16 and 17 (also referred to above, in the representative example section, as 1A, 1B, 1C, 1D, and 1E, respectively) from the above procedures were: (1) 12.65 nM for Compound 14, (2) 1.0 nM for Compound 15, (3) >1500 nM for Compound 16, (4) 12.54 nM for Compound 17, and (5) 1.86 nM for Compound 18.

The CB2 Ki for Compounds 14, 15, 18, 16 and 17 from the above procedures were: (1) >1800 nM for Compound 14, (2) >1500 nM for Compound 15, (3) >1500 for Compound 16, (4) >1800 nM for Compound 17, and (5) >1500 nM for Compound 18.

Compound 15 had a CB2 Ki of>1500 nM.

While the present invention has been. described in conjunction with the specific embodiments set forth above, many alternatives, modifications and variations thereof will be apparent to those of ordinary skill in the art. All such alternatives, modifications and variations are intended to fall within the spirit and scope of the present invention.

What is claimed is:

1. A compound of the formula: 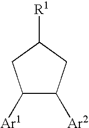
or the pharmaceutically acceptable salts and solvates thereof, wherein:
Ar¹ is 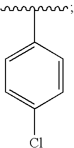
Ar² is 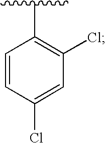
R¹ is —(CH₂)ₘ—X—(CH₂)ₙ—R²;
X is selected from the group consisting of: —NH—, —O—, —C(O)— and —S(O)₂—;
R² is 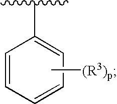
R³ is selected from the group consisting of: halo and —CN;
m is 0 to 4;
n is 0 or 1; and
p is 1, 2, or 3.

2. The compound of claim 1 wherein said compound is selected from the group consisting of:

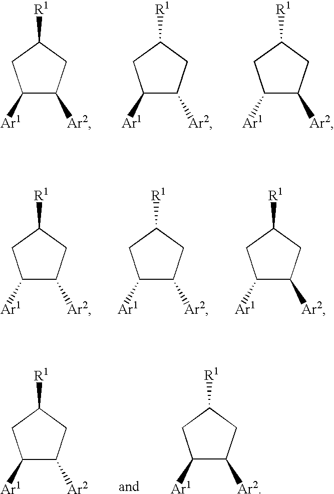

3. The compound of claim 1 wherein said compound is:

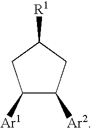

4. The compound of claim 1 wherein X is selected from the group consisting of: —NH— and —O—.

5. The compound of claim 1 wherein X is —NH—.

6. The compound of claim 1 wherein X is —O—.

7. The compound of claim 1 wherein R³ is selected from the group consisting of: F and —CN.

8. The compound of claim 1 wherein R³ is —CN.

9. The compound of claim 1 wherein p is 1 or 2.

10. The compound of claim 1 wherein p is 1 when R³ is —CN, and p is 2 when R³ is F.

11. The compound of claim 1 wherein R² is selected from the group consisting of:

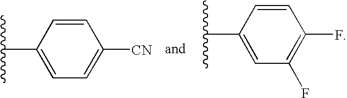

12. The compound of claim 1 wherein R² is

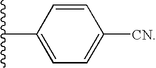

13. The compound of claim 1 wherein R² is

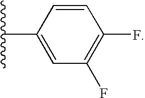

14. The compound of claim 1 wherein m is 1.

15. The compound of claim 1 wherein n is 0.

16. The compound of claim 1 wherein n is 1.

17. The compound of claim 1 wherein R¹ is selected from the group consisting of:

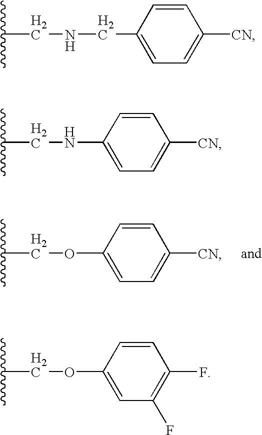

18. The compound of claim 1 wherein R¹ is:

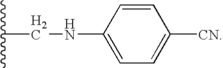

19. The compound of claim 1 selected from the group consisting of:

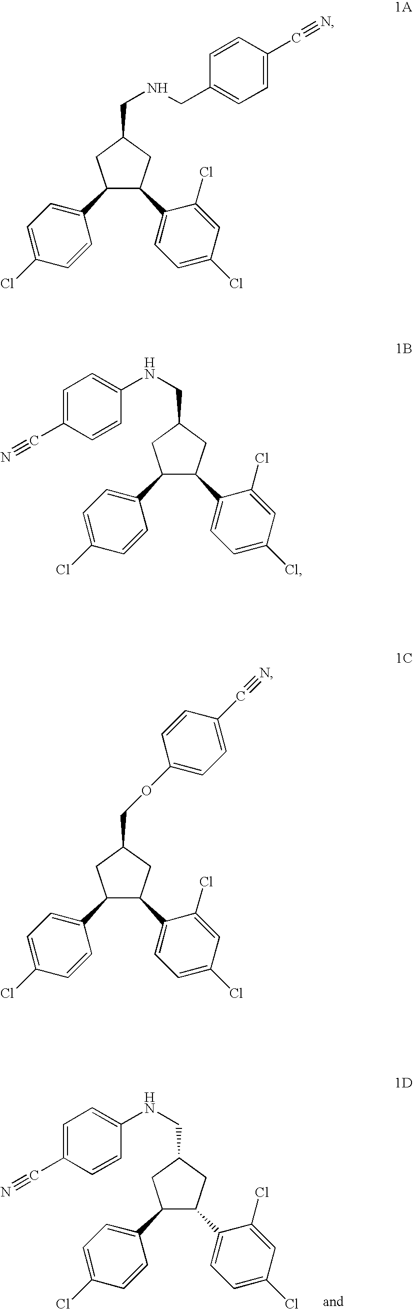

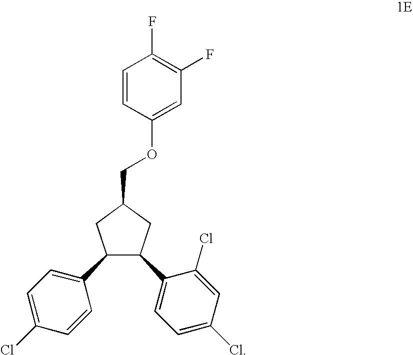

20. The compound of claim 19 wherein said compound is 1A.

21. The compound of claim 19 wherein said compound is 1B.

22. The compound of claim 19 wherein said compound is 1C.

23. The compound of claim 19 wherein said compound is 1D.

24. The compound of claim 19 wherein said compound is 1E.

25. A pharmaceutical composition comprising at least one compound of claim 1 and a pharmaceutically acceptable carrier.

26. A pharmaceutical composition comprising at least one compound of claim 19 and a pharmaceutically acceptable carrier.

27. A method of treating a cannabinoid receptor mediated disease in a patient in need of such treatment, said treatment comprising administering to said patient an effective amount of at least one compound of claim 1.

28. A method of treating a cannabinoid receptor mediated disease in a patient in need of such treatment, said treatment comprising administering to said patient an effective amount of at least one compound of claim 19.

29. A method of treating a cannabinoid receptor mediated disease in a patient in need of such treatment, said treatment comprising administering to said patient an effective amount of a compound of claim 1 in combination with at least one other pharmaceutically active agent.

30. A method of treating a cannabinoid receptor mediated disease in a patient in need of such treatment, said treatment comprising administering to said patient an effective amount of a compound of claim 19 in combination with at least one other pharmaceutically active agent.

31. A method of treating the metabolic syndrome in a patient in need of such treatment, said treatment comprising administering to said patient at least one compound of claim 1.

32. A method of treating the metabolic syndrome in a patient in need of such treatment, said treatment comprising administering to said patient at least one compound of claim 1, and an effective amount of at least one other pharmaceutically active ingredient.

33. A method of treating atherogenic dyslipidemia, in a patient in need of such treatment, said treatment comprising administering to said patient an effective amount of at least one compound of claim 1.

34. A method treating high triglycerides, low HDL cholesterol and high LDL cholesterol, in a patient in need of such treatment, said treatment comprising administering to said patient an effective amount of at least one compound of claim 1.

35. A method of treating high triglycerides, low HDL cholesterol and high LDL cholesterol, in a patient in need of such treatment, said treatment comprising administering to said patient an effective amount of at least one compound of claim 1, in combination with at least one cholesterol lowering agent.

36. A method of treating high triglycerides, low HDL cholesterol and high LDL cholesterol in a patient in need of such treatment, said treatment comprising administering to said patient an effective amount of at least one compound of claim 1, and an effective amount of at least one cholesterol lowering agent selected from the group consisting of: ezetimibe, the combination of ezetimibe and simvastatin, lovastatin, simvastatin, pravastatin, atorvastatin calcium, and rosuvastatin calcium.

37. The method of claim 36 wherein said cholesterol lowering agent is selected from the group consisting of: ezetimibe, and the combination of ezetimibe/simvastatin.

38. A method of treating a disease or condition, in a patient in need of such treatment, wherein said disease or condition is selected from the group consisting of: neuroinflammatory disorders, cognitive disorders, psychosis, addictive behavior, gastrointestinal disorders, and cardiovascular conditions, said treatment comprising administering to said patient an effective amount of a compound of claim 1.

39. A method of treating a disease or condition, in a patient in need thereof, wherein said disease or disorder is selected from the group consisting of: neuroinflammatory disorders, cognitive disorders, psychosis, addictive behavior, gastrointestinal disorders, and cardiovascular conditions, said treatment comprising administering to said patient an effective amount of at least one compound of claim 1, in combination with at least one other pharmaceutically active ingredient.

40. A method of treating abdominal obesity in a patient in need thereof, comprising administering to said patient an effective amount of at least one compound of claim 1.

41. A method of treating abdominal obesity in a patient in need thereof, comprising administering to said patient an effective amount of at least one compound of claim 1, in combination with at least one other pharmaceutically active ingredient.
